(19) United States
(12) Patent Application Publication
(10) Pub. No.: US 2025/0051773 A1
(43) Pub. Date: Feb. 13, 2025

(54) DNAZYME AND USE THEREOF

(71) Applicant: SHANGHAI CANGYU BIOTECHNOLOGY CO., LTD, Shanghai (CN)

(72) Inventors: Hongzhou GU, Shanghai (CN); Qiao ZHANG, Shanghai (CN); Kai XIA, Shanghai (CN); Fuyou LI, Shanghai (CN)

(73) Assignee: SHANGHAI CANGYU BIOTECHNOLOGY CO., LTD, Shanghai (CN)

(21) Appl. No.: 18/853,908

(22) PCT Filed: Apr. 7, 2023
(86) PCT No.: PCT/CN2023/086801

§ 371 Date: Oct. 3, 2024

(30) Foreign Application Priority Data
Apr. 8, 2022 (WO) PCT/CN2022/085696

(51) Int. Cl. C12N 15/113; C12P 19/34

(52) U.S. Cl. CPC C12N 15/113; C12P 19/34; C12N 2310/127

(57) ABSTRACT

The present disclosure provides a DNAzyme and use thereof. The present disclosure also provides a system, comprising one or more catalytic domains and one or more substrate domains.

### BACKGROUND OF THE INVENTION

Currently, DNA nanotechnology, the field of biomedical research such as knock-in, has a wide demand for Single strand DNA (ssDNA), especially for long Single strand DNA (>100 bases). However, due to the limitation of chemical synthesis methods, the synthesis of long single-stranded DNA is difficult. Furthermore, in practical applications, the methods known in the art all have problems of low yield, high cost, and the like.

### SUMMARY OF THE INVENTION

The present disclosure provides a system of DNAzyme for preparing long Single strand DNA. The system may robustly generate cleavage products with customizable 5′ and/or 3′ termini. The products of the present disclosure may display robust effects in excising a series of oligos of different lengths with high yield and accuracy.

In one aspect, the present disclosure provides a system, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and G on 3′ side of said cleavage site.

In one aspect, the present disclosure provides a nucleic acid, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and G on 3′ side of said cleavage site.

In one aspect, the present disclosure provides a vector, comprising the system of the present disclosure and/or the nucleic acid of the present disclosure.

In one aspect, the present disclosure provides a cell, comprising the system of the present disclosure, the nucleic acid of the present disclosure and/or the vector of the present disclosure.

In one aspect, the present disclosure provides a composition, comprising the system of the present disclosure, the nucleic acid of the present disclosure, the vector of the present disclosure and/or the cell of the present disclosure.

In one aspect, the present disclosure provides a kit, comprising the system of the present disclosure, the nucleic acid of the present disclosure, the vector of the present disclosure, the cell of the present disclosure, and/or the composition of the present disclosure.

In one aspect, the present disclosure provides a method of preparing a product, comprising providing the system of the present disclosure, the nucleic acid of the present disclosure, the vector of the present disclosure, the cell of the present disclosure, the composition of the present disclosure and/or the kit of the present disclosure.

In one aspect, the present disclosure provides a product prepared according to the method of the present disclosure.

In one aspect, the present disclosure provides a combination, comprising providing a condition comprising about 1 to 2 mM Zn²⁺, and about 5 to 20 mM Mn²⁺.

In one aspect, the present disclosure provides a method of preparing a product, comprising providing the combination of the present disclosure and providing 5′ nucleic acid cutter and 3′ nucleic acid cutter.

In one aspect, the present disclosure provides a product prepared according to the method of the present disclosure.

In one aspect, the present disclosure provides a method of nucleic acid detection, comprising providing the product of the present disclosure.

In one aspect, the present disclosure provides a method of sequencing, comprising providing the product of the present disclosure.

In one aspect, the present disclosure provides a method of genetic engineering, comprising providing the product of the present disclosure.

In one aspect, the present disclosure provides a method of data storage, comprising providing the product of the present disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCES

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates PECAN—a biotechnological method assisted by paired-end cutting with DNAzymes to produce arbitrary DNA sequences. a, Schematic of PECAN protocol. b, Sequence and secondary structure of 13PD1, a previously reported DNAzyme. After screening, this DNA enzyme was chosen as DNAzyme 1. Its cleavage site was highlighted by a scissor. C. Sequence and secondary structure of II-R1—another previously reported DNAzyme, and its mutants II-R2&R3 that were identified through a reselection in this work. The mutated nucleotides were labeled. II-R2 and II-R3 can be selectively used as DNAzyme 2. d, Kinetic characterization of the 5′ and 3′ self-cutters through denaturing PAGE (dPAGE) analysis. Filled and hollow arrowheads refer to the uncleaved and cleaved DNA, respectively. e, Plot of the fraction of DNA cleaved vs time for 13PD1 and its mutants. Data was extracted from gels in d. The k_{obs} and 1-h yield values show that the single nucleotide identity at the 3′ of the cleavage site (default T as in 13PD1) moderately affects the cleavage speed but not the 1-h cleavage yield (>85% for T, A, G, and C). f, Plot of the fraction of DNA cleaved vs time for II-R1, R2, and R3. Data was extracted from gels in d and FIGS. 8&9. The summarized k_{obs} and 1-h yield values indicate that II-R2&R3 are much more robust than II-R1. For II-R2, in 1 h an over 85% yield was gained as the single nucleotide identity at the 5′ of the cleavage site to be a G, A, or C, but not T (63±3% yield). The T-less-effectiveness-in-yield issue of II-R2 was resolved by replacement of II-R2 with II-R3, which cleaves right after a T with a 1-h yield of 88±2%. The standard deviation (S.D.) of yields in e&f was generated from three replicate assays. Check mark designates each DNAzyme suitable for PECAN.

FIG. 2 illustrates Comparison of PECAN oligos with CS oligos. a, Comparison by dPAGE analysis on a representative 71-nt DNA. Marker 1&2: 20 nt ladder and 50 nt ladder, respectively. b. Comparison by monoisotopic spectroscopy on the 71-nt DNA. The observed molecular weight (MW) of the CS 71mer (21,667.9 Da) and the PECAN 71mer (21,668.2 Da) matched well with the calculated value (21,667.5 Da). Besides the major mass peak, many weak peaks (circled by dash line) appeared around it for the CS 71mer. c, Comparison by dPAGE analysis on the representative 65-nt and 69-nt oligos. d, Sequence and secondary structure information as well as full-length purity of the three representative oligos. The comparison between CS and PECAN was based on each NGS with >1,000,000 reads. e, Workflow of RNA in situ imaging with PIP DNA. f, Detection of HER2 expression in MCF-7 and CCNB1 expression in SK-BR-3 cells by CS and PECAN PLP. The detected mRNAs were shown as dots. Nuclei stained with DAI were shown in blue. g, Statistics plot of the detected RCPs per cell for HER2 and CCNB1 by CS and PECAN PLP. Two-tailed paired Student t-test P-values indicate statistical significance (****P<0.0001). h, Detection of multi-mRNA expression in HER2 positive breast cancer FFPE tissue sections by CS and PECAN PLP. Merged were the detection of the expression of HER2, MK167, ESR1, and PGR. An enlarged view (boxed area) was shown on the right for each tissue section. Scale bar: 100 μm. i, Statistics plot of the detected RCPs per cell for HER2, MK167, ESR1, and PGR in tissue section by CS and PECAN PLP.

FIG. 3 illustrates Producing LASSO probes by PECAN for analysis of RNA splicing variants. a, Alternative splicing patterns of S100P and CYP24A1 in 971. and NL cells. b, Schematic of single target capture with LASSO probes. LASSO l&2 were designed specifically for S100P and CYP24A1 tv1, respectively. c, Quality check of the LASSO probes produced by PECAN. M3: 200 nt ladder. LASSO 1: 335 nt. LASSO 2: 550 nt. d, Gel analysis of the targeted amplification products post capture. LASSO 1&2 were programmed to capture a 401 bp and a 749 bp fragment within S100P and CYP24A1 tv1 transcripts, respectively. An unexpected band was referred by *. The NL samples were used as negative controls. M4: 100 bp ladder. e, Sequencing of the captured targets. Transcripts of S100P and CYP24A1 tv1 in 97L were confirmed. Also revealed in 97L was another isoform of CYP24A1, named CYP24A1 tvX1, which contains a 172-bp insertion between Exon 11 & 12. f, Screening a series of hepatoma cell lines for splice isoforms of CYP24A1 with PECAN LASSO 2.

FIG. 4 illustrates Producing gene-sized HDRTs by PECAN for precise and efficient genome editing. a. Schematic of the HDR-guided knock-in. The CRISPR/Cas9 system was used to create dsDNA breaks at specific sites of genomic DNA. During HDR, either dsDNA or ssDNA with homology arms can serve as the template (HDRT) to insert exogenous sequence into genomic DNA. b, Comparison of two 1,570-nt DNAs produced by commercialized strandase kit (Kit) and PECAN through dPAGE analysis. + & − refer to sense and anti-sense strand, respectively. M3: 200 nt ladder. e, Confocal microscopy imaging of endogenous fluorescence in Hek293T cells with HDRT of dsDNA, Kit ssDNA, and PECAN ssDNA for K1. The fused protein tags include mEGFP on TUBA1B (tubulin alpha 1b), mCherry on CLTA (clathrin light chain A), mEGFP on FBL (fibrillarin), and BFP on RAB11A (Rab protein 11A). The apparent off-targeting was pointed out by arrowheads. d, Representative flow cytometry plots showing the off-target efficiency for dsDNA, Kit+, and PECAN+ HDRTs in Hek293T cells. The experiments were conducted with Cas9 and without sgRNA. e, Comparison of the apparent K1 efficiency for dsDNA, Kit+, and PECAN+ HDRTs in Hek293T and H9 cells. f, Confocal microscopy imaging of endogenous fluorescence in Hek293T cells with KI of double tags by dsDNA and PECAN+ HDRTs. In each panel, visualization of the mEGFP and mCherry tags were shown in the upper and lower right, respectively, and the merged view was shown in the left. g, A reconstructed 31) image (i) showing the colocalization of TDNs with microtubules in a Hek293T cell. The position of the TDNs-Cy5 was determined by 3D optical sectioning (ii-iv). h, Colocalization of TDNs-Cy5 with three-color confocal microscopy in Hek293T cells under different phases of the cell cycle. Scale bar: 10 μm.

FIG. 5 illustrates Producing ˜7,000mer oligos by PECAN for data storage. a, Schematic of the PECAN oligo-based data storage system. b. Comparison of characteristics of current DNA-based storage systems.

FIG. 6 illustrates I-R3 as an imperfect DNAzyme 1 for PECAN. a, Sequence and secondary structure of I-R3, a previously reported DNAzyme and a potential DNAzyme 1 for PECAN. Its cleavage site was highlighted by a scissor. b, Analysis of I-R3's cleavage activity for all 16 combinations of the two nucleotides at \hat{ }AG-3′ by dPAGE. c, Analysis of I-R3's cleavage activity for the mutation of \hat{ }AG-3′ to \hat{ }N-3′ by dPAGE. d. Re-examination of I-R3's cleavage site for sequences of \hat{ }NG-3′. All gels were stained for band analysis. The yield and S.D. were generated from three replicate assays

FIG. 7 illustrates Programmability of the first nucleotide in the stem downstream of \hat{ }N-3′ for 13PD1 and its mutants. a-d, Schematic of the covariation for 13PD1, 13PD1-A (\hat{ }A-3′), 13PD1-G (\hat{ }G-3′), & 13PD1-C (\hat{ }C-3′) and analysis of the respective cleavage activity by dPAGE. All gels were stained for band analysis. The yield and S.D. were generated from three replicate assays.

FIG. 8 illustrates Reselection on II-R1. a, Kinetic characterization of II-R1 (5′-G\hat{ }) and its mutants II-R1-A (5′-A\hat{ }), II-R1-T (5′-T\hat{ }), & II-R1-C (5′-C\hat{ }) by dPAGE. b, Schematic of the creation of the degenerate DNA libraries for II-R1 reselection. Based on the reselection, we built the consensus sequence and secondary structural model for the class 11 DNAzyme. Gray, black, and red nucleotides designate conservation of at least 75%, 90%, and 97%, respectively. Nucleotides less conserved are represented by circles. Green shading denotes base pairs supported by covariation. R refers to purine. c, Sequences of II-R1 and -R2&3 mutants that can cleave faster. Dot denotes the identical nucleotide. Gray shading denotes sequences that form the P1&2 stems. The arrowhead points to the cleavage site. And the 5′ nucleotide at the cleavage site for II-R2&3 is highlighted.

FIG. 9 illustrates Characterization of II-R2&3. a, Kinetic characterization of II-R2-T (5′-T\hat{ }) through dPAGE analysis. b-e, Schematic of the covariation for II-R2-G (5′-G\hat{ }), II-R2-A (5′-A\hat{ }), II-R2-C (5′-C\hat{ }), & II-R3 (5′-T\hat{ }) and analysis of the respective cleavage activity by dPAGE. The gels in b-e were stained for band analysis. The yield and S.D. were generated from three replicate assays.

FIG. 10 illustrates identifying the optimal metal ion condition for robust co-hydrolysis of II-R2/3 and 13PD1. a, Testing the activity of II-R2-G and 13PD1 in buffers (pH 7.0 at 23° C.) containing various concentrations of Zn²⁺ and Mn²⁺. For II-R2-G alone, the default metal requirement is 2 mM Zn²⁺; for 13PD1, the optimal metal condition is 1 mM Zn²⁺ and 20 mM Mn²⁺. After test and analysis, 1 mM Zn²⁺ and 5 mM Mn²⁺ were eventually chosen as the optimal metal ion concentrations for the two enzymes in one pot to robustly cleave each self. With 1 mM Zn²⁺ and 5 mM Mn²⁺, to ensure over 90% cleavage yields for both enzymes, incubation at 37° C. for 2 h is suggested. b, Confirming the optimal condition of 1 mM Zn²⁺ and 5 mM Mn²⁺ on II-R2-A, II-R2-C, II-R3, and 13PD1-A/G/C.

FIG. 11 illustrates Examples of programming DNAzyme sequences into customized oligos for PECAN production. a, paring 13PD1 with II-R2-C to produce a 71 mer oligo. b, paring 13PD1 with II-R3 to produce a 1,390mer oligo.

FIG. 12 illustrates Examples of collecting ss-phagemid precursors for PECAN production of customized oligos. a, gathered phagemid precursors in petri dishes. From top to bottom, the precursors carry a 71 nt, 1,390 nt, and 6.790 nt customized sequence. The samples were weighed after lyophilization. b, denaturing gels showing the nearly completely release of the target oligos from the recombinant phagemid precursors after PECAN processing. Note that only a tiny small portion of the DNA samples in a was processed by PECAN and showcased in b.

FIG. 13 illustrates Error distribution in CS and PECAN oligos. a-c, Comparison between the CS and PECAN 65mer, 69mer, and 71mer oligo, respectively. Analysis was based on NGS data. Mut (Indels) x refers to oligo sequences with x number of mutated (inserted or deleted) nucleotides.

FIG. 14 illustrates Quality check of the PLPs produced by PECAN. a, dPAGE analysis of the PLPs used for RNA imaging in cells. Ml: 20 nt ladder. b, dPAGE analysis of the PLPs used for RNA imaging in tissue samples. c, Sequences of all PLPs used in this study.

FIG. 15 illustrates Detection of multi-mRNA expression in HER2 positive breast cancer FFPE tissue sections. a, Detection by CS PLP. b, Detection by PECAN PLP. HER2: red; MK167: cyan; ESR1: yellow; PGR: green. Nuclei stained with DAPI were shown in blue. Merged and enlarged views (boxed area) of the four RNAs were shown in FIG. 2h. Scale bar: 100 μm.

FIG. 16 illustrates Analysis of the sequencing data of the captured fragments. a, Sequences captured by PECAN LASSO 1 from 97L. b&c, Sequences captured by PECAN LASSO 2 from 97L.

FIG. 17 illustrates Quality check of the PECAN and Kit HDRTs by dPAGE. M3: 20 nt ladder. Arrowheads point to the speculated highly-structured-and-non-denaturable DNAs.

FIG. 18 illustrates Cell viability with PECAN vs dsDNA HDRT. a, Marked decrease in the number of live cells under microscopy after electroporation with dsDNA HDRT, as comparing to PECAN HDRT. b, Plot of the cell viability vs the DNA amount used for KI with dsDNA, PECAN+, and PECAN− HDRTs. The experiment was conducted in Hek293T cells.

FIG. 19 illustrates Measurement of the on-target GFP frequency at the TUBA1B locus by a ddPCR assay. a, Schematic of the measurement strategy. RPP30 was chosen as a reference gene to test the ddPCR conditions. b, 1D fluorescence amplitude plots of ddPCR amplification products. The droplets yielding close-to-background signals were represented by gray dots. c, The calculated concentration (copies/μl) of the detected gene fragment by ddPCR, d, The estimated on-target integration of mEGFP into TUBA1B for PECAN and Kit HDRTs.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term complementary used here generally refer to forming base pairs between nucleic acids. Oligonucleotides and their analogs hybridize by hydrogen bonding, which includes Watson-Crick, Hoogsteen or reversed Hoogsteen hydrogen bonding, between complementary bases. Generally. nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. Artificially or naturally modified nitrogenous bases can be involved. For example, pseudoisocytosine (J), or 5-methylcytosine (5mC) will hydrogen bond to G. “Complementary” refers to the base pairing that occurs between two distinct nucleic acids or two distinct regions of the same nucleic acid. “Specifically hybridizable” and “specifically complementary” are terms that indicate a sufficient degree of complementarity such that stable and specific binding occurs between a nucleic acid (or its analog) and another nucleic acid target (e.g., DNA or RNA). The nucleic acid or analog may, but need not have, 100% complementarity to its target sequence to be specifically hybridizable. For example, a nucleic acid molecule specifically binds another nucleic acid molecule if a sufficient amount of the nucleic acid molecule forms base pairs or is hybridized to its target nucleic acid molecule to permit detection of that binding (such as at least 90%, 91%, 92%, 93%, 94%, 95%, 96%, 97%, 98%, 99%, or 100% complementary).

The term catalytic nucleic acids used here generally refer to nucleic acid molecules that are capable of catalyzing a specific chemical reaction (such as oxidative cleavage or hydrolytic cleavage, for example, phosphodiester hydrolytic cleavage, nucleoside excision, phosphorylation (or de-phosphorylation), ligation, or other reactions). Catalytic nucleic acids include ribozymes (catalytic RNA or RNAzymes),

The term deoxyribozymes DNAzymes) used here generally refer to DNA molecules capable of catalyzing specific chemical reactions. DNAzymes may catalyze nucleic acid cleavage (such as oxidative cleavage or hydrolytic cleavage, for example, phosphodiester hydrolytic cleavage), nucleoside excision, phosphorylation (or de-phosphorylation), ligation, or other reactions. DNAzymes may or may not have one or more unnatural chemical modifications on the nitrogenous bases and/or backbone thereof. DNAzymes (catalytic DNA or deoxyribozymes), and other natural or unnatural, modified, or unmodified nucleic acid molecules.

The term single-stranded nucleic acid used here generally refer to a nucleic acid that only includes a single polymer strand (e.g., the nucleic acid polymer strand does not form non-covalent bonds with another nucleic acid polymer), such as single-stranded DNA (ssDNA). The nucleic acid molecule can be single-stranded in full (e.g., ssDNA formed through melting a double-stranded DNA molecule) or in part (e.g., a ssDNA region formed through damage and/or enzymatic activity).

The term vector used here generally refer to a nucleic acid molecule as introduced into a host cell, thereby producing a transformed, transfected, or transduced host cell. A vector may include nucleic acid sequences that permit it to replicate in the host cell, such as an origin of replication.

In one aspect, the present application provides a system, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and 6 on 3′ side of said cleavage site.

For example, the catalytic domains may be catalytic domains of DNAzyme. The substrate domains may be the nucleic acid product to be prepared, or substrate domains may be a part the nucleic acid product to be prepared, or substrate domains may be a 5′ part the nucleic acid product to be prepared. Not all DNAzyme may generate any user-defined 5′ termino of the substrate domain or nucleic acid product. For example, it is expected that changing the 5′ termino of substrate domain would lead to catalytic domains of DNAzyme losing its cleavage ability. The reason may be that 5′ termino of the substrate domain possesses conserved nucleotides, and the conserved nucleotides may be a “scar” on nucleic acid product. It is surprising to find the 13PD, one of many DNAzyme, may generate the substrate domain or nucleic acid product having 5′ termino of A, C, or G, other than T.

For example, said system may comprise one or more catalytic nucleic acids, and said catalytic nucleic acids comprise one or more said catalytic domains. For example, wherein said system may comprise one or more substrate nucleic acids, and said substrate nucleic acids comprise one or more said substrate domains.

For example, one or more said catalytic nucleic acids and one or more said substrate nucleic acids are separate and/or conjugated. For example, wherein one or more said catalytic nucleic acids and one or more said substrate nucleic acids are linked via one or more nucleotide containing any kind of bases.

For example, said system further may comprise one or more binding domains, and said binding domains flank and/or is within said catalytic domains and/or said substrate domains.

For example, said catalytic nucleic acids comprise one or more binding domain A, said substrate nucleic acids comprise one or more binding domain B, and said binding domain A is capable of binding to said binding domain B.

For example, said catalytic nucleic acids comprise binding domain A-5 on 5′ side of said catalytic domains and binding domain A-3 on 3′ side of said catalytic domains, said substrate nucleic acids comprise binding domain B-5 on 5′ side of said substrate domains and binding domain B-3 on 3′ side of said substrate domains, and said binding domain A-5 is complementary to said binding domain B-3 and/or said binding domain A-3 is complementary to said binding domain B-5.

For example, said 13PD comprise 13PD1, 13PD2, 13PD3, 13PD4, and/or the mutant thereof. For example, said catalytic domains comprise sequence of SEQ ID NO: 17. For example, said catalytic domains comprise nucleic acid hydrolysis activity. Furthermore, catalytic domain can be engineered by in vitro selection for high sequence recognition specificity, single-base-level reaction site specificity, customizability, stability, and/or low cost.

For example, said substrate domains comprise sequence of SEQ ID NO: 18 (actgcn, wherein n is a, c or g).

For example, said substrate domains comprise A, C, or G on 3′ end of said substrate domains. For example, 3′ end of said substrate domains is A, C, or G. For example, 3′ end of said substrate domains is not T.

In one aspect, the present application provides a nucleic acid, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and G on 3′ side of said cleavage site.

For example, said nucleic acid further may comprise one or more binding domains, and said binding domains flank and/or is within said catalytic domains and/or said substrate domains.

For example, said nucleic acids comprise one or more binding domain A flanking said catalytic domains, said nucleic acids comprise one or more binding domain B flanking said substrate domains, and said binding domain A is capable of binding to said binding domain B.

For example, said nucleic acids comprise binding domain A-5 on 5′ side of said catalytic domains and binding domain A-3 on 3′ side of said catalytic domains, said nucleic acids comprise binding domain B-5 on 5′ side of said substrate domains and binding domain B-3 on 3′ side of said substrate domains, and said binding domain A-5 is complementary to said binding domain B-3 and/or said binding domain A-3 is complementary to said binding domain B-5.

For example, said 13PD comprise 13PD1, 13PD2, 13PD3, 13PD4, and/or the mutant thereof. For example, said catalytic domains comprise sequence of SEQ ID NO: 17. For example, said catalytic domains comprise nucleic acid hydrolysis activity. For example, said substrate domains comprise sequence of SEQ ID NO: 18 (actgen, wherein n is a, c or g).

For example, said substrate domains comprise A, C, or G on 3′ end of said substrate domains. For example, 3′ end of said substrate domains is not T.

In one aspect, the present application provides a vector, comprising the system of the present application and/or the nucleic acid of the present application.

In one aspect, the present application provides a cell, comprising the system of the present application, the nucleic acid of the present application and/or the vector of the present application.

In one aspect, the present application provides a composition, comprising the system of the present application, the nucleic acid of the present application, the vector of the present application and/or the cell of the present application.

In one aspect, the present application provides a kit, comprising the system of the present application, the nucleic acid of the present application, the vector of the present application, the cell of the present application, and/or the composition of the present application.

In one aspect, the present application provides a method of preparing a product, comprising providing the system of the present application, the nucleic acid of the present application, the vector of the present application, the cell of the present application, the composition of the present application and/or the kit of the present application.

In one aspect. the present application provides a product prepared according to the method of the present application. For example, the product to be prepared may comprise nucleic acid. For example, the product to be prepared may comprise DNA. RNA and/or PNA.

For example, said product may comprise nucleic acid. For example, the product may comprise at least 5, 6, 7, 8, 9, 10, 11, 12, 13, 14, 15, 20, 25, 30, 35, 40, or 45, 50, 100, 1000, 10000, 100000 or 1000000 nucleotides. For example, k-mers may be substrings of length k contained within a biological sequence. the sequence AGAT would have four monomers (A, G, A, and T), three 2-mers (AG, GA, AT), two 3-mers (AGA and GAT) and one 4-mer (AGAT).

In one aspect, the present application provides a combination, comprising providing a condition comprising about 1 to 2 mM Zn²⁺, and about 5 to 20 mM Mn²⁺.

For example, combination may comprise providing a condition comprising about 1 mM Zn²⁺, and about 5 mM Mn²⁺.

For example, combination may comprise about 1 mM Zn²⁺, and about 5 mM Mn²⁺. For example, combination may comprise about 1 mM Zn²⁺, and about 5 mM Mn²⁺, and the pH of the combination may be about 6-8. For example, combination may comprise about 1 mM Zn²⁺, and about 5 mM Mn²⁺, and the pH1 of the combination may be about 7. Further, in some examples. temperature can include at least about 4° C., 10° C., 15° C., 20° C., 25° C., 30° C. 32° C., 36° C., 37° C., 38° C., 40° C., 41° C., 42° C., 45° C., 50° C. 55° C. 60° C., 65° C., 70° C., 75° C., 80° C., 90° C., or 95° C. or about 4-90° C., 15-50° C., 20-40′C, 32° C.-42° C., 36° C.-42° C., 38° C.-42° C., 41° C.-42° C., 37° C.-95° C., 37° C.-60° C. or 40° C.-60° C. Further, in some examples, reaction time can include at least about at least 15 min (such as at least 15 min, 18 min, 20 min, 25 min, 30 min, 40 min, 60 min, 1.5 hr, 2 hr, 4 hr, 6 hr, 8 hr, 10 hr, 12 hr, 18 hr, or overnight or about 15 min-overnight, 20 min-overnight, 40 min-overnight, 2 hr-overnight, 20 min-18 hr, 40 min-18 hr, 2 hr-18 hr, 6 hr-18 hr, or 8 hr-12 hr about 20 min or 2 hr).

In one aspect, the present application provides a method of preparing a product, comprising providing the combination of the present application and providing 5′ nucleic acid cutter and 3′ nucleic acid cutter. For example, the 5′ nucleic acid cutter and 3′ nucleic acid cutter may be in the combination simultaneously. For example, the 5′ nucleic acid cutter and 3′ nucleic acid cutter may cleave each self in said combination simultaneously.

For example, said 5′ nucleic acid cutter may comprise DNAzyme 1 capable of generating 3′ cleavage product.

For example, said 5′ nucleic acid cutter is on 5′ side of said product. For example, said 5′ nucleic acid cutter and said product are separate and/or conjugated. For example, said 5′ nucleic acid cutter and said product are on same chain.

For example, said 5′ nucleic acid cutter may comprise 13PD and mutant thereof. For example, said 5′ nucleic acid cutter may comprise 13PD1, 13PB2, I-R3, and mutant thereof.

For example, said 3′ nucleic acid cutter may comprise DNAzyme II capable of generating 5′ cleavage product.

For example, said 3′ nucleic acid cutter is on 3′ side of said product. For example, said 3′ nucleic acid cutter and said product are separate and/or conjugated. For example, said 3′ nucleic acid cutter and said product are on same nucleic acid.

For example, said 3′ nucleic acid cutter may comprise II-R1 and mutant thereof.

For example, said 3′ nucleic acid cutter may comprise II-R1a, II-R1b, II-R1c, II-R1d, and mutant thereof.

Further, the one or more 5′ nucleic acid cutter and the one or more 3′ nucleic acid cutter may be used at a variety of ratios, such as at least 1:1, 1:2,1:3, 1:4,1:5, or 1:10, or about 1:1-1:5 or 1:1-1:2 or about 1:2 5′ nucleic acid cutter to 3′ nucleic acid cutter.

In one aspect, the present application provides a product prepared according to the method of the present application.

For example, said product may comprise nucleic acid.

In one aspect, the present application provides a method of nucleic acid detection, comprising providing the product of the present application.

For example, the product of the present application may be used as padlock probes (PLP). For example, PLP in combination with rolling-circle amplification (RCA) can generate clonally amplified rolling-circle products (RCPs) at high density in preserved tissue and cells for detection.

For example, the method of the present application is performed in vitro, ex vivo, in vivo, and/or in cellulo.

In one aspect, the present application provides a method of sequencing, comprising providing the product of the present application.

For example, the product of the present application may be used as long padlock or long-adapter single-strand oligonucleotide (LASSO) probe. For example, a sample's genome information may be analyzed by targeted sequencing.

For example, the method of the present application is performed in vitro, ex vivo, in vivo. and/or in cellulo.

In one aspect, the present application provides a method of genetic engineering, comprising providing the product of the present application.

For example, the product of the present application may be used as HDR. template. For example, sequence insertion may use exogenous DNA donors as templates for homology-directed repair (HDR).

For example, the method of the present application is performed in vitro, ex vivo, in vivo, and/or in cellulo.

In one aspect, the present application provides a method of data storage, comprising providing the product of the present application.

For example, the product of the present application may be used for DNA and/or RNA-based storage.

For example, the method of the present application is performed in vitro, ex vivo, in vivo, and/or in cellulo.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); sa, amino acid(s); nt, nucleotide(s); i.m., intramuscular(ly); i.p., intraperitoneal(ly); s.c., subcutaneous(ly); and the like.

### Methods

Reselection on II-R1 for the 3′ self-cutter. Based on the II-R1 sequence, we synthesized several degenerate DNA libraries (IDT), including

(SEQ ID NO: 1)
5′-pGAGTGCTACGAACGTAGGAGCATCTTTGGCGTACAAGCGAAGCTT
GTACGCTAGGGGAATAAATCTTTGGGTGCCTACGTTCGTAGGACTC,
(SEQ ID NO: 2)
5′-pGAGTGCTACGAACGTAGAAGCATCTTTGGCGTACAAGCGAAGCTT
GTACGCTAGGGGAATAAATCTTTGGGTGTCTACGTTCGTAGGACTC,
(SEQ ID NO: 3)
5′-pGAGTGCTACGAACGTAGTAGCATCTTTGGCGTACAAGCGAAGCTT
GTACGCTAGGGGAATAAATCTTTGGGTGACTACGTTCGTAGGACTC,
(SEQ ID NO: 4)
5′-pGAGTGCTACGAACGTAGCAGCATCTTTGGCGTACAAGCGAAGCTT
GTACGCTAGGGGAATAAATCTTTGGGTGGCTACGTTCGTAGGACTC,
and
(SEQ ID NO: 1)
(5′-pGAGTGCTACGAACGTAGGAGCATCTTTGGCGTACAAGCGAAGCTT
GTACGCTAGGGGAATAAATCTTTGGGTGCCTACGTTCGTAGGACTC),

with a degeneracy of 0.18 at each of the underlined nucleotides (41-46 nt in total). This yielded initial (G0) DNA pools containing DNA molecules with an average of seven to eight mutations relative to the intact II-R1 precursor. We also designed a pair of primers 1

(5′-pGAGTGCTACGAACGT (SEQ ID NO: 5))
and
2
(5′-AAAAAAAAAAAAAAA (SEQ ID NO: 6)/spacerC18/
GAGTCCTACGAACGT (SEQ ID NO: 7))

for selective amplification. A spacer modification (spacerC18) was included in primer 2 to stall polymerase extension on its A1s tail, so that the sense and anti-sense strand of the PCR products can be differentiated by length through dPAGE.

For each library, to start the reselection, we ligated 200 pmol of the G0 population with CircLigase (200 Units, EpiCentre) at 60° C. for 2 hours in a 200 μL mixture containing 1× CircLigase reaction buffer, 50 μM ATP, and 2.5 mM MnCl₂. We precipitated the reaction products with 100% ethanol and purified the monomeric DNA circles by using 10% dPAGE. We then incubated the recovered circular DNAs at 37° C. for 30 min in 100 μL of the selection buffer containing 50 mM HEPES (pH 7.0 at 23° C.), 100 mM NaCl, 10 mM MgCl₂, and 2 mM ZnC₂. We separated the cleaved DNAs (linear) from the uncleaved ones (circular) by 10% dPAGE. The recovered linear DNAs were re-ligated with CircLigase (30 Units, EpiCentre) in a 30 μL mixture containing 1× CircLigase reaction buffer, 50 μM ATP, and 2.5 mM MnCl₂. By 10% dPAGE, we then isolated the circular DNAs. Using them as the template, we conducted PCR amplification with 100 pmol each of primers 1 and 2. According to the length difference, we separated the sense (91 nt) from the anti-sense strand (106 nt) of the PCR products on 10% dPAGE. We recovered the sense DNAs to rebuild the DNA pool for next-round selection.

To select for II-R1 mutants with robust activity in cleaving DNA, we shortened the incubation time of DNA pools with the selection buffer from 30 min (G0-G3) to 5 min (G4-G5), then to 1 min (G6-G7). We picked up the G3 pool and deep sequenced it using NGS. According to the sequencing data of G3 of the five libraries, we rebuilt the consensus sequence and secondary structural model of the class II Zn²⁺-dependent deoxyribozymes. Besides, we collected ˜100 clones (TOPO TA Cloning Kit, Invitrogen) from G7 and individually sequenced them. This generated 38 unique sequences, which were screened to identify mutants (II-R2-G, II-R2-A, II-R2-C, and II-R3) of II-R1 with desired activity and sequence-generality (a combinatorial usage of the four mutants to achieve generality) at the 5′ of the cleavage site.

DNAzyme cleavage assay. We designed DNAzymes either in the unimolecular or bimolecular form for the cleavage assay. The DNAzymes were incubated in the corresponding reaction buffer, i.e., a buffer of 50 mM HEPES (pH 7.0 at 23° C.), 100 mM NaCl, 10 mM MgCl₂, 20 mM MnCl₂, and 1 mM ZnCl₂ for 13PD1 and its variants, as well as a buffer of 50 mM HEPES (pH 7.0 at 23° C.), 100 mM NaCl, 10 mM MgCl₂, and 2 mM ZnCl₂ for II-R1, R2&3. The incubation was conducted at 37° C. for minutes to hours, depending on the purpose of the assay. To map the cleavage site of DNAzymes, the samples were incubated for 1 h and then mixed with (v/v: 1/1) the loading buffer (90% formamide, 30 mM EDTA, 0.025% bromophenol blue, 0.025% xylene cyanol) to stop the reaction. To characterize the kinetics of DNAzymes, the samples were pipetted out and mixed with (v/v: 1/1) the loading buffer to stop the reaction at different time points (0 s, 20 s, 40 s, 1 min, 2 min, 5 min, 10 min, 20 min, 40 min, and 1 h).

We then used denaturing PAGE to analyze the cleavage. For kinetic assays conducted on bimolecular constructs, we labeled the substrate DNA strand with a 5′ FAM and detected the fluorescence signal on dPAGE gels by a Typhoon FLA9500 scanner. We extracted the information of fraction cleaved vs time from the gels to calculate the k_{obs} and yield of each DNAzyme. Values for the k_{obs} were established by using the following equation: fraction cleaved=FCₘₐₓ(1−e^{-ki}), where k=k_{obs} and FCₘₐₓ=maximum of fraction cleaved. For mapping assays, we stained the gels by SYBR Gold, scanned them with a Bio-rad ChemiDoc MP Imaging System, and analyzed the data with the ImageQuant software.

### PECAN Protocol.

Design of the pseudogene of the customized ssDNA. We initiated the protocol by designing DNA construct that carries a pair of self-cleaving DNAzymes at the 5′ and 3′ end of the customized ssDNA for phagemid recombination. Nucleotide identity at the 5′ and 3′ end of the customized sequence determines the corresponding DNAzymes that are chosen. The rules of the 5′ DNAzyme are 13PD1 for 5′ T, 13PD1-A for 5′ A, 13PD1-G for 5′G, and 13PD1-C for 5′ C.

The Bold base part is the catalytic domain and the substrate domain, and the sequence is conserved;

The underlined base part is the base complementary pairing region, and the sequence can be changed;

Label|Italic base is the cleavage site
(1) The deoxyribozyme catalytic domain and the substrate domain are separated and function in the form of two DNA strands:

13PD1 catalytic sequence:
(SEQ ID NO: 8)
5′GTCGCCATCTCTTCTATACCGGGCAACTATTGCCTCGTCATCGCTATTTTCTGCGA
TAGTGAGTCGTATTA-3′
13PD1 substrate sequence:
(SEQ ID NO: 9)
5′-TAATACGACTCACTATACTGC|TGAAGAGATGGCGAC-3′
13PD1-A catalytic sequence:
(SEQ ID NO: 8)
5′GTCGCCATCTCTTCTATACCGGGCAACTATTGCCTCGTCATCGCTATTTTCTGCGA
TAGTGAGTCGTATTA-3′
13PD1-A substrate sequence:
(SEQ ID NO: 10)
5′TAATACGACTCACTATACTGC|AGAAGAGATGGCGAC-3′
13PD1-C catalytic sequence:
(SEQ ID NO: 8)
5′GTCGCCATCTCTTCTATACCGGGCAACTATTGCCTCGTCATCGCTATTTTCTGCGA
TAGTGAGTCGTATTA-3′
13PD1-C substrate sequence:
(SEQ ID NO: 11)
5′-TAATACGACTCACTATACTGC|CGAAGAGATGGCGAC-3′
13PD1-G catalytic sequence:
(SEQ ID NO: 8)
5′GTCGCCATCTCTTCTATACCGGGCAACTATTGCCTCGTCATCGCTATTTTCTGCGA
TAGTGAGTCGTATTA-3′
13PD1-G substrate sequence:
(SEQ ID NO: 12)
5-TAATACGACTCACTATACTGC|GGAAGAGATGGCGAC-3′
(2) The deoxyribozyme catalytic domain and substrate domain are on one
DNA chain and function in the form of one DNA chain:
13PD1:
(SEQ ID NO: 13)
5′GTCGCCATCTCTTCTATACCGGGCAACTATTGCCTCGTCATCGCTATTTTCTGCGA
TAGTGAGTCGTATTATTTTAATACGACTCACTATACTGC|TGAAGAGATGGCGAC-3′
13PD1-A:
(SEQ ID NO: 14)
5′GTCGCCATCTCTTCTATACCGGGCAACTATTGCCTCGTCATCGCTATTTTCTGCGA
TAGTGAGTCGTATATTTTAATACGACTCACTATACTGC|AGAAGAGATGGCGAC-3′
13PD1-C:
(SEQ ID NO: 15)
5′GTCGCCATCTCTTCTATACCGGGCAACTATTGCCTCGTCATCGCTATTTTCTGCGA
TAGTGAGTCGTATTATTTTAATACGACTCACTATACTGC|CGAAGAGATGGCGAC-3′
13PD1-G:
(SEQ ID NO: 16)
5′GTCGCCATCTCTTCTATACCGGGCAACTATTGCCTCGTCATCGCTATTTTCTGCGA
TAGTGAGTCGTATTATTTTAATACGACTCACTATACTGC|GGAAGAGATGGCGAC-3′

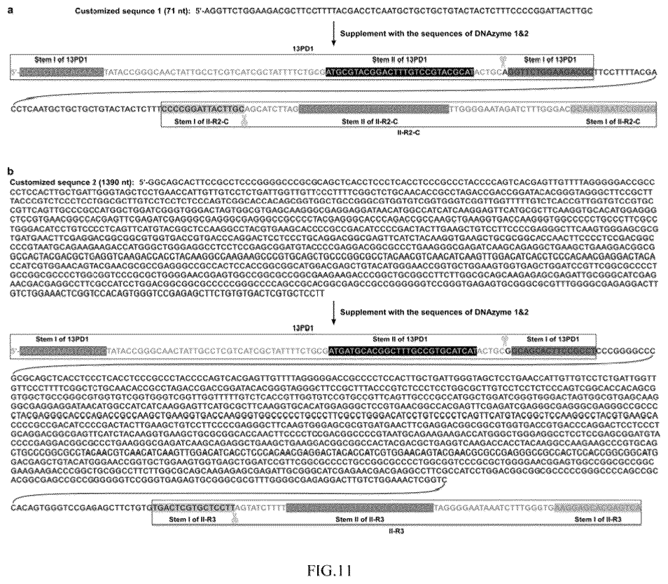

The choices of the 3′ DNAzyme are II-R2-G for 3′ G, II-R2-A for 3′ A, II-R3 for 3′ T, and II-R2-C for 3′ C. Once the pair of DNAzymes was determined, we supplemented their sequences to the two ends of the customized ssDNA, such that the supplements can self-fold into the secondary structure of the corresponding DNAzymes by recruiting end-sequences of the customized ssDNA as part of their structure. This was achieved by programming ˜15 nt of the supplemented sequences for complementation with ˜15 nt of end-sequences of the customized ssDNA to form a critical supportive stem of DNAzymes (See FIG. 11 for examples and details). The supplementation of DNAzyme sequences expanded the length of customized DNA to over 200 nt. We treated such long DNA constructs as double-stranded pseudogene fragments for gene synthesis.

Synthesis of the pseudogene of the customized ssDNA. The length of our designed pseudogenes in this study ranges from ˜230 bp to ˜7000 bp. Such large DNA fragments can be efficiently assembled in yeast by one-step assembly of series of overlapping synthetic ssDNAs (˜100-150 nt in length) with a linearized plasmid vector in a simple transformation event (Gibson assembly).²⁶ This technique has been commercialized by many companies to synthesize “gene fragments”. Instead of a normal plasmid vector, herein we requested a local company (GeneRay Biotech, Shanghai) to assemble our pseudogene fragments with the p3024 phagemid vector during transformation. The products were delivered to us in the form of a recombinant p3024 phagemid (˜5 μg) with pseudogene inserted. The recombinant p3024 can later be used to produce phagemid particles carrying ssDNA phagemid with the aid of a helper phage.

Production of ssDNA phagemid particles. For each phagemid amplification, we thawed 100 μl of JM109 competent cells (Sangon Biotech) on ice and mixed the cells with 1-10 ng recombinant p3024 phagemid for 30-min incubation on ice. Then we heat-pulsed the sample in a 42° C. water bath for 45 sec and incubated it on ice again for 2-3 min. We added 900 μl of the LB medium to the sample and shook it with a speed of 220 r/min at 37° C. for 1 h. The sample was then centrifuged at 5,000 r/min for 5 min. We removed 900 μl of the supernatant, resuspended the cells in the left-over medium, and plated the medium on a preheated (37° C.) LB agar plate containing ampicillin (100 μg/ml). We placed the plate upside down and incubated it at 37° C. for 12 h. On the second day, we picked up single colonies (usually two to four) and screened them by sequencing for the one(s) with the correct transformation of the recombinant p3024.

For typical culturing, we grew the recombinant p3024 transformed single colonies in 15 ml LB medium with ampicillin (100 μg/ml) at 37° C. overnight. We then inoculated each of the four bottles of 300 ml 2×YT medium (16.0 g/l tryptone, 10.0 g/l yeast extract, 5.0 g/l NaCl, 5 mM MgCl₂, pH 7.0) with 3 ml of the overnight culture, and shook them at 37° C. with a speed of 250 r/min. We monitored OD₆₀₀ of the culture every 30 min. As the OD₆₀₀ value reached ˜0.4-0.5, we added VCSM13 helper phage to the culture with a MOI of 20 (ratio of phage to cells, 50 μl of 3×10¹⁰ phage/μl VCSM13 phage stock to 300 ml of cell culture with an OD₆₀₀ value of ˜0.5 (2.5×10⁸ cells/ml)). 30 min later, we added kanamycin (final concentration of 70 μg/ml) to the culture to select for infected cells. We continued to shake the culture for 4.5 h, and then collected the culture for centrifugation at 4000 r/min for 15 min at 4° C. We transferred the supernatants to clean bottles and supplemented with PEG 8000 (40 g/1) and NaCl (30 g/l). After vigorous agitation (stirring) to dissolve the powders, we incubated the mixture on ice for 30 min and then centrifuged it at 5,000 ref for 30 min at 4° C. We discarded the supernatants and resuspended the phagemid pellets in 10 ml Tris (10 mM, pH 8.5), which was further centrifuged at 16,000 ref for 10 min at 4° C. to remove any bacterial residue. We collected the resulting supernatants that contain pure recombinant p3024 phagemid particles.

Extraction of ssDNA phagemid. The ssDNA phagemid was extracted from phagemid particles by stripping the proteins coat. We gently mixed 2× volume (relative to the volume of collection of phagemid particles) of NaOH (0.2 M, with 1% SDS) with the phagemid collection by swirling. We incubated the mixture at room temperature for 3 min and then gently mixed 1.5× volume of KOAc (3 M, titrated with glacial acetic acid to pH 5.5) with the sample by inversion. Then we further incubated the sample in ice water bath for 10 min and centrifuged it at 16,000 rcf for 30 min at 4° C. We collected the supernatants, mixed them with 2× volume of 100% ethanol, and incubated it in ice water bath for 30 min. We spun the mixture again at 16,000 ref for 30 min at 4° C., collected the pellets (the recombinant p3024 ssDNA) and washed them with 75% ethanol to remove additional salts. We left the pellets in the air for 10-min drying and then lyophilized them for weighing and storage. Typically we obtained ˜3-8 mg recombinant p3024 ssDNA from 1.2 l culture prepared in shake flask. As we performed the culturing in a 10 l laboratory fermenter, we routinely gained ˜0.4-1 g recombinant p3024 ssDNA.

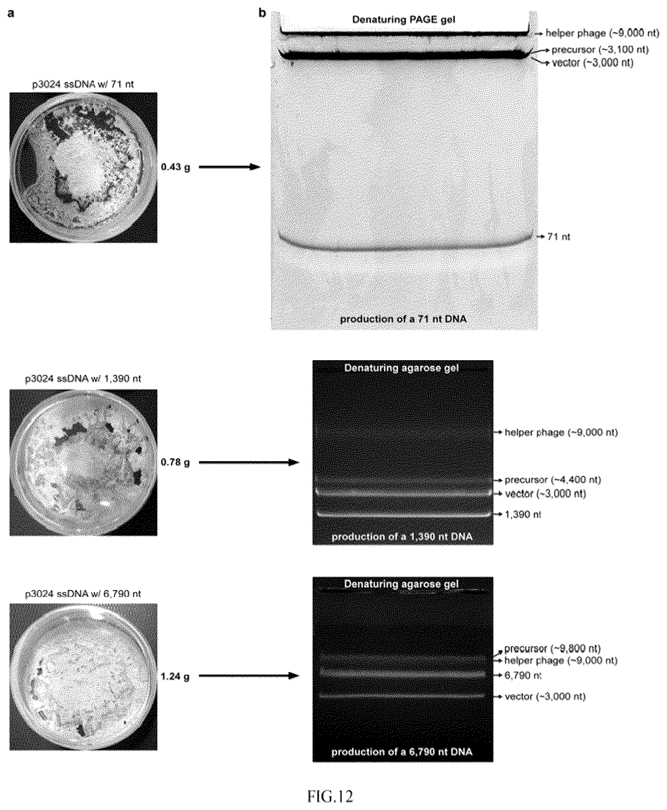

Release of the customized ssDNA b, induced DNAzyme-cutting. The customized ssDNA was amplified via the p3024 vector. To release it from the vector, we induced the programmed DNAzyme pairs flanking the customized ssDNA to self-cleave. We dissolved the recombinant p3024 ssDNA (a final concentration of 100 nM) in a buffer containing 50 mM HEPES (pH 7.0 at 22° C.), 100 mM NaCl, and 10 mM MgCl₂. Then we conducted an annealing protocol of 90° C. for 3 min. 75° C. for 5 min, 60° C. for 5 min, 45° C. for 5 min, and 22° C. for 5 min. We added equal volume of a second buffer containing 50 mM HEPES (pH 7.0 at 23° C.), 100 mM NaCl, 10 mM MgCl₂, 10 mM MnCl₂, and 2 mM ZnCl₂ to the sample, and incubated it at 37° C. to allow the DNAzyme-catalyzed DNA processing for hours (2-4 h). We mixed the reaction products vigorously with 3× volume of 100% ethanol (pre-cooled at 4° C.) and centrifuged the mixture at 16,000 ref for 30 min at 4° C., We collected the pellets, washed them with 75% ethanol (pre-cooled at 4° C.) to remove residual salt, centrifuged the sample again at 16,000 ref for 7 min at 4° C., and then resuspended the pellets in a 1× denaturing loading buffer (40 mM Tris, 40 mM Borate, 6 M Urea, 0.5 mM EDTA, pH 7.2, 10% (w/v) sucrose, 0.05% SDS, 0.01% (w/v) Bromphenol Blue, 0.01% (w/v) Xylene Cyanole). For customized ssDNAs less than 1,000 nt long, we chose denaturing PAGE to purify them; for customized ssDNAs over 1,000 nt, we purified them by denaturing agarose gels (See FIG. 12 for examples). Alternatively, we used the protocol of anion exchange chromatography for large scale (mg-g) purification of ssDNA.

RNA in situ detection. For cell samples grown on slides, we used 0.1 M HCl to permeabilize the cells. The samples were then washed with 0.05% (v/v) Tween-20 in 1× diethyl pyrocarbonate treated phosphate buffered saline (DEPC-PBS) for 2 min. For human breast cancer formalin-fixed paraffin-embedded (FFPE) tissue sections obtained from the Pathology Department of Quanzhou First Hospital Affiliated to Fujian Medical University (China), we incubated them at 60° C. in the oven for 30 min, and then washed them twice with ddH₂O for 15 min and 10 min consecutively. The slides were rehydrated with a series of decreasing concentrations of ethanol (100%, 95%, 70%) twice each for 2 min, and treated with diethyl pyrocarbonate treated H₂O (DEPC-H₂O) for 5 min and 1×DEPC-PBS for 2 min, followed by fixation using 4% paraformaldehyde for 10 min. The samples were washed with 1×DEPC-PBS for 2 min. Permeabilization was conducted in 0.1 M HCl solution containing 0.1 mg/ml pepsin at 37° C. for 30 min. Dehydration was then performed with an ethanol gradient of 70%, 85% and 100% for 1 min each. The use of human tissue material is in accordance to the requirements of the ethical committee of Huaqiao University.

After pretreatment, the reaction area was demarcated using Secure-Seal hybridization chamber (Thermo Scientific) or ImmEdge Pen (Sigma-Aldrich). We added 0.1 μM padlock probe (chemically synthesized with 5′ phosphorylation or prepared by PECAN) in hybridization buffer (6× saline sodium citrate (SSC, Sigma-Aldrich), 10% formamide) to the reaction area for incubation at 37° C. for 2 h. The samples were then washed three times with 2×SSC supplemented with 20% formamide and three times with DEPC-PBS. Next, we incubated the samples at 37° (for 1 h with a reaction mixture containing 0.5 U/μl SplintR ligase, 1×SplintR ligase reaction buffer (New England Biolabs), 50% (v/v) glycerol, 1 U/μl RiboLock RNase Inhibitor, and 0.2 μg/L BSA in DEPC-120. After three times wash with DEPC-PBS, we added 1 μM primer DNA in rolling-circle amplification (RCA) buffer to the samples and incubated them at 37° C. for 30 min, followed by three times wash with DEPC-PBS. Then the RCA reaction was carried out in a mixture of 1 U/μl phi29 DNA polymerase, 1×phi29 DNA polymerase reaction buffer, 1 mM dNTPs, 5% (v/v) glycerol, and 0.2 μg/μl BSA in DEPC-H₂O at 30° C. overnight. The samples were washed three times with a mixture of 1×DEPC-PBS, 2×SSC, and 20% formamide. After that, we applied 0.1 μM detection probe to the samples and kept them at 22° C. for 30 min. Excess probe was removed by three times wash with DEPC-PBS. The samples were then stained with 0.5 μg/mL DAPI in SlowFade Gold Antifade Mountant (Thermofisher) before imaging. For analysis of pictures taken by fluorescent microscopy, we used Ilastik (https://www.ilastik.org) to identify nuclei and CellProfiler (https://www.cellprofiler.org/) to identify RCP dots.

### Analysis of RNA Splicing.

Design and construction of LASSO probes. According to the known transcripts information, we chose the appropriate sites (25-35 nt) on the transcripts of interest for specific recognition by LASSO probes, in a fashion that the length of the captured sequence in the transcripts was set to be ˜400-800 nt, spanning two to three exons. A pair of the primers that bind to the recognition sites was then covalently joined in an inverted-molecular-probe way by filling in ˜300-500 scrambled nucleotides as a long adaptor between them. The resulted LASSO probes were subjected to PECAN protocol for ssDNA production.

cDNA Preparation. We isolated total RNA from ˜10⁷ cells each sample by using TRIzol Reagent (Invitrogen) according to the included user guide. To synthesize the cDNA, we started with 2 μg total RNA as the template for reverse transcription by a commercialized kit (PrimeScript™ RT reagent Kit with gDNA Eraser, Takara). The collected cDNA was quantified by a NanoDrop spectrophotometer (Thermo Scientific).

Capturing by LASSO probes. We mixed 1-10 fmol of LASSO probes with 100 ng cDNA in 20 μl of 1× Ampligase buffer (EpiCentre). To ensure the hybridization of LASSO probes with cDNA. we heated the mixture to 98° C. for 5 min. and slowly cooled it down to 56° C. at a rate of 1° C. per minute. For probe extension and ligation, 0.6 U Phusion polymerase, 5 U Ampligase (EpiCentre) and 3 pmol dNTP were added to the mixture. The sample was further incubated at 56° C. for 60 min, 72° C. for 20 min, 95° C. for 3 min, and then cooled on ice. To eliminate linear cDNA in the mixture, we digested the reaction products with 0.5 μl of Exonuclease 1 (20 units/μl) and 0.5 μl of Exonuclease III (100 units/μl) at 37° C. for 30 min. After that, the exonuclease in the sample was inactivated by incubation at 90° C. for 10 min.

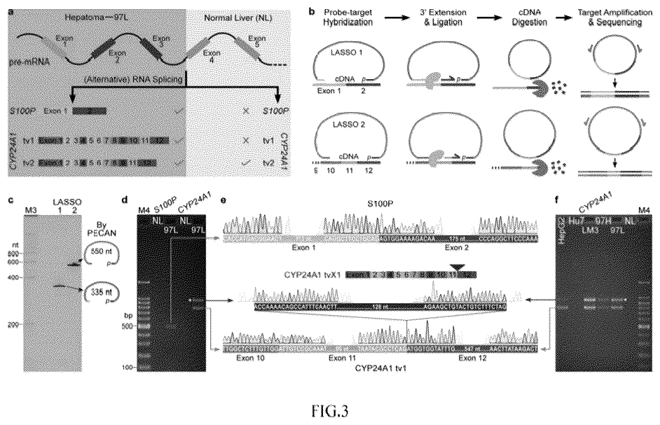

Detection and sequencing of the captured products. For each captured product, we used a pair of primers targeting the probes' adaptor region to amplify the captured sequences (see FIG. 3b). About 5 μl of the captured products was used as template for amplification, and 30 PCR cycles were performed. The PCR products were analyzed by agarose gel electrophoresis and sequenced after gel purification.

### Gene Knock-In.

Cell culturing. HEK 293T cells were maintained in DMEM (Hyclone) glutamax medium supplemented with 10% fetal bovine serum (FBS, Gibco), 100 units/ml penicillin, and 100 g/ml streptomycin (Beyotime). H9 hESCs were cultured on matrigel-coated plates (Gibco) in mTeSRTM plus basal medium (Stemcell Technologies) with 10% FBS. All cells were kept in a humidified atmosphere of 5% CO₂ at 37° C.

In-vitro transcription and purifcation of sgRNA. The DNA templates for sgRNAs were ordered from Generay Biotechnology (China). Transcription was performed in a 1×Transcription buffer (40 mM Tris pH 8.0, 20 mM MgCl₂, 5 mM DTT, and 2 mM spermidine) supplemented with 2 mM NTP, 80 pmol DNA template, and 10 U T7 RNA polymerase. Samples were incubated at 37° C. for 3 h. The sgRNA products were later purified by 8% denaturing PAGE gels, aliquoted and stored at −80° C., Expression and purification of Cas9. The recombinant S. pyogenes Cas9 (pMJ915) construct was purchased from Addgene (Plasmid No.: 69090), and transformed into E. coli BL21 (DE3) competent cells for Cas9 expression. pMJ915 transformed single colonies were grown in 10 ml LB medium with 100 μg/ml ampicillin at 37° C. and shaken at a speed of 250 rpm overnight. The cells were then inoculated into 112×YT medium for culturing at 37° C. until OD₆₀₀ reached ˜0.4-0.5. The culture was cooled down to 15° C. for 2 h. Protein expression was induced by the supplement of 0.1 mM Isopropyl β-D-1-thiogalactopyronoside (IPTG). After culturing at 15° C. for 16 h, cells were pelleted by centrifugation at 5,0(0) rpm for 15 min at 4° C. After removal of the supernatants, cells were resuspended in the lysis buffer of 300 mM NaCl, 30 mM Tris-4Cl (pH 8.0), and 0.01% β-mercaptoethanol, and lysed by using a high-pressure homogenizer (EmulsiFlex-C3, Avestin). The cell lysates were collected and centrifuged at 16,000 rpm for 30 min at 4° C. Then the collected supernatants were incubated with Ni Sepharose™ 6 fast flow beads (GE Healthcare) at 4° C. for 1 h. We used the buffer containing 500 mM NaCl, 30 mM Tris-HCl (pH 8.0), 0.01% β-mercaptoethanol, and 40 mM Imidazole to wash the beads to remove nonspecifically bound proteins. After washing, Cas9 protein was eluted with a buffer of 150 mM NaCl, 30 mM Tris-HCl (pH 8.0), 0.01% β-mercaptoethanol, and 220 mM imidazole. The elution was further purified by using SSS (AKTA Purifier system. GE Healthcare) cation-exchange chromatography. The resulted Cas9 protein was quantified by using the BCA protein assay kit (Abeam) and stored in the buffer of 25 mM HEPES (pH 7.4), 30 mM NaCl, 3 mM DTT, and 20% glycerol at −80° C.

Preparation of dsDNA HDRT. All of the dsDNA HDRT were prepared by PCR amplification using the corresponding plasmids as templates (see Supplementary Table I and 2 for sequences). The amplification was performed in a 50 μl solution mixed by 25 μl 2×Phanta Flash Master Mix (Vazyme), 2 μl of each primer (10 μM), 1 μl of the plasmid template (10 ng/μl), and 20 μl ddH₂O. The products were purified by using TaKaRa MiniBEST DNA Fragment Purification Kit Ver.4.0 (Takara Biotechnology). The DNA samples were resuspended in ddH₂O, and quality-checked by 1% agarose gel electrophoresis. The concentration of each dsDNA HDRT was determined by Nanodrop (Thermo Fisher).

Supplementary Table 1

| | | Addgene | | | |
|---|---|---|---|---|---|
| | | # of | | | |
| HDRT | Target | plasmid | | | SEQ ID |
| Template | gene | template | Primer sequence (5′ to 3′) | | NO |
| mEGFP- | TUBA1B | #87421 | Forward | AGGAAGGCCTTCCCGG | 19 |
| TUBA1B | | | Reverse | CATAGTTATTGGCAGCATCTTCCTTG | 20 |
| CLTA-mCherry | CLTA | #112017 | Forward | GGCAGCACTTCCGCCTCCC | 21 |
| | | | Reverse | AAGGAGCACGAGTCACACAGAAGC | 22 |
| FBL-mEGFP | FBL | #87427 | Forward | GAGAAAAACAGCCCAAAGCCCTG | 23 |
| | | | Reverse | AGCAAAATGGCGACCACAACAA | 24 |
| RAB11A-BFP | RAB11A | #112014 | Forward | CTAGGAGTTCCAGGACTCAGTTT | 25 |
| | | | Reverse | GACGATGTGGGAGAAGGCAGTCCG | 26 |

SUPPLEMENTARY TABLE 2

| Name | Sequence (5′ to 3′) |
|---|---|
| mEGFP-TUBA1B HDRT (1,570 nt) | SEQ ID NO: 27 |
| FBL-mEGFP HDRT (1,480 nt) | SEQ ID NO: 28 |
| CLTA-mCherry HDRT (1,390 nt) | SEQ ID NO: 29 |
| RAB11A-BFP HDRT (1,355 nt) | SEQ ID NO: 30 |

Preparation of ssDNA HDRT by exonuclease digestion (Kit). We use the same protocol in Preparation of dsDNA HDRT to obtain the dsDNA HDRT firstly. During PCR amplification, we used a 5′ phosphorylated primer to generate dsDNA, thus one strand of which can be digested later. We treated the purified dsDNA HDRT with the Guide-it™ Long ssDNA Production System (Takara Biotechnology) according to the manufacture's recommendations. Specifically, the DNA strand with a 5′ phosphate was digested by Strandase Mix A&B. After enzymatic digestion, the remaining ssDNA products were purified by using NucleoSpin Gel and PCR Clean-up Kit (Macherey-Nagel). Using this strategy. we prepared both the sense and anti-sense ssDNA HDRT. The ssDNA samples were quality-checked by 1% agarose gel electrophoresis. The concentration of each ssDNA HDRT was quantified by Nanodrop.

Cell electroporation. HEK 293T and H9 hESCs cells were transfected by using the Neon Transfection System 10 μl Kit (Thermo Fisher) according to the manufacture's recommendations. For each homologous recombination assay, 1.5 μg of Cas9 protein and 360 ng of sgRNA were added to the Resuspention Buffer R to a final volume of 2 μl. The samples were incubated at 23° C. for 10 min to allow the pre-assembly of Cas9 RNPs. 2.5 μg of the HDR donor DNA (dsDNA, PECAN ssDNA, or Kit ssDNA) dissolved in 8 pI Buffer R was then supplemented with the RNPs. The total 10 μl samples were mixed with 2 μl Buffer R containing 1.8×10⁵ HEK 293T cells or 5×10⁵ H9 hESCs cells. The cells were electroporated immediately by using the optimized program. For HEK 2931 cells, the program was set at 1,200 V, with a 20 ms pulse width for 2 pulses. After electransfection, HEK 293T cells were seeded in 500 μl pre-warmed DMEM culture media on a 24-well plate. For H9 hESCs, cells were firstly dissociated into a single-cell suspension by using Accutase™ (Gibco). The subsequent electrophoration program was set at 1,100 V, with a 20 ms pulse width for 2 pulses. After electransfection, H9 hESCs cells were seeded in 500 μl pre-warmed mTeSRTM Plus culture media on a Matrigel-coated 24-well plate. The electroporated cells were cultured for 2-3 days and then dissociated into single cells for FACS analysis.

Flow cytometry and analysis. To determine the percentage of mEGFP-positive, mCherry-positive, or mBFP-positive cells, after electroporation and culturing, HEK 293T and 119 hESCs cells were individually analyzed on a BD LSRFortessa flow cytometry instrument. Cell sorting was performed on a Motlo Astrios EQ 4. Flow cytometry data analysis and figure preparation was conducted with the FlowJo software (FlowJo LLC).

Cell imaging. For confocal microscopy imaging, HEK 293T and 19 hESCs cells were individually grown in 35 mm glass dishes (Cellvis) after electroporation or cell sorting. Live cells were imaged on a TCS SP8 STED 3× microscope (Leica) at 63× and 63×3 magnification.

### Example 1 DNAzymes for PECAN Development

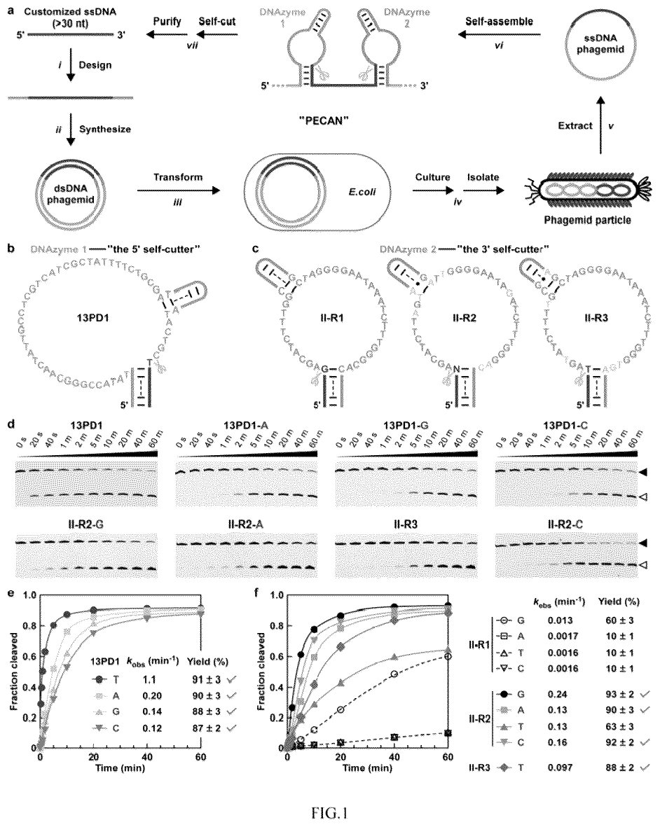

Despite of many efforts in selection and optimization, lack of broad sequence generality has been still unresolved for DNAzyme-guided specific cleavage of DNA. Taking that into consideration. we managed to circumvent rather than overcome this generality issue to conceive PECAN. By programming distinctive DNAzyme 1&2 as the 5′&3′ self-cutter of the target oligo, respectively, we expected to attenuate the stringency in sequence generality required from each enzyme for scar-free oligo production (FIG. 1a). Conceivably, with such a scheme we only need DNAzyme 1 capable of tolerating 3′ sequence and DNAzyme 2 capable of tolerating 5′ sequence at their corresponding cleavage sites.

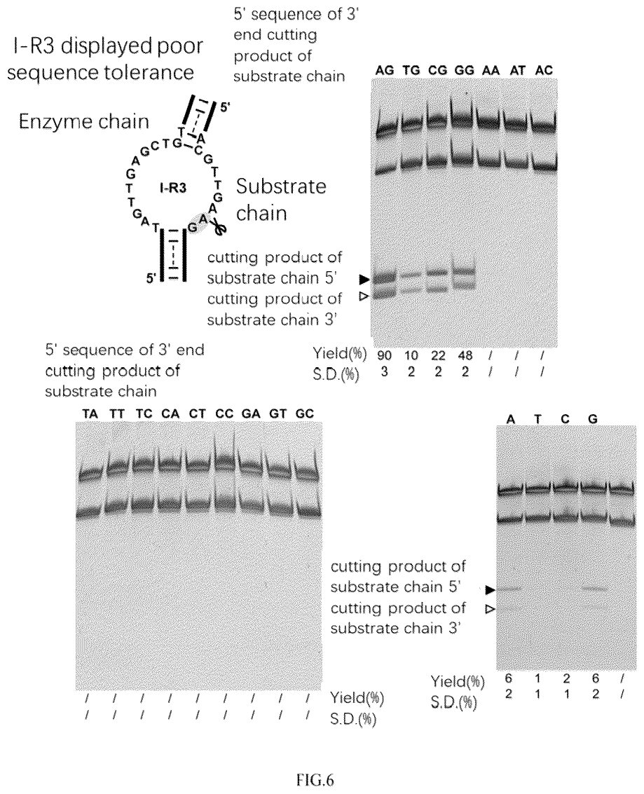

By searching through the pool of known DNA-cleaving DNAzymes, we quickly nailed down 13PD1 and I-R3 as the potential candidates for DNAzyme 1, because both had been reported to site-specifically hydrolyze DNA and possess only one (for 13PD1, FIG. 1b) or two (for I-R3, FIG. 6a) conserved nucleotides (nts) at the 3′ of the cleavage site (hereafter referred by \hat{ }). At 37° C., the two enzymes' robustness is reflected by an observed rate constant (k_{obs}) value of ˜1 min⁻¹ and >90% yield in 5 min.

Point-mutation experiments confirmed the preference of \hat{ }T>A>G>C-3′ for 13PD1, as shown by the 10-fold decrease in k_{obs} from \hat{ }T to \hat{ }C (FIG. 1d-e). However, such loss in catalytic speed can be simply compensated by moderate extension of the reaction time, e.g., from 5 min to 1 h, to achieve an almost lossless yield (>85%) in DNA cleavage for \hat{ }A, \hat{ }G, or \hat{ }C mutant of 13PD1 (FIG. 1d-e). Contrarily, I-R3 displayed poor sequence tolerance at the conserved \hat{ }AG-3′, with irrecoverable yields in cleavage for most of the mutants (FIG. 6b-d).

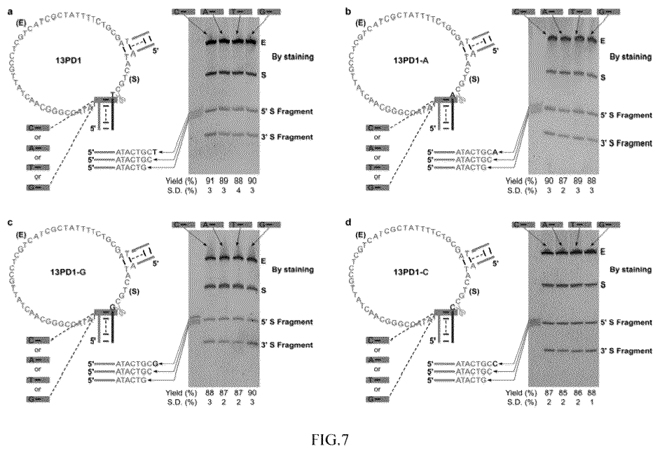

Nucleotides downstream of \hat{ }T-3′ in 13PD1 are buried inside an apparant stem according to the reported secondary structure model of this enzyme (FIG. 1b). Generally, they can be programmed to any identity, as long as the base complementation in the stem is maintained. Consistently, comprehensive covariation on the first base-pair downstream of \hat{ }T-3′ revealed unaltered yields (>85% in 1 h) for 13PD1 and \hat{ }A, \hat{ }G, \hat{ }C mutants (FIG. 7). Collectively, we determined that 13PD1 can serve as DNAzyme 1 to effectively generate 3′ cleavage products with customizable 5′ termini (\hat{ }NN-3′. N refers to any nucleotide).

Next, we moved to DNAzyme 2 and considered II-R1 as the candidate because it hydrolyzes DNA right after a 5′ stem (FIG. 1c). Nevertheless, tests displayed that this enzyme also has nucleotide preference (5′-G>A/T/C\hat{ }), and even for the preferred 5′-G\hat{ }, II-R1 cleaved only at a k_{obs} value of 0.013 min⁻¹ or a yield of 60% in 1 h (FIG. 1f, FIG. 8a). To simultaneously improve II-R1's 5′ sequence tolerance and hydrolysis activity, we preseted the 5′ nucleotide to G, A, T, or C, and created the respective degenerate DNA library to reselect for mutants that may cleave faster (see Methods, FIG. 8b).

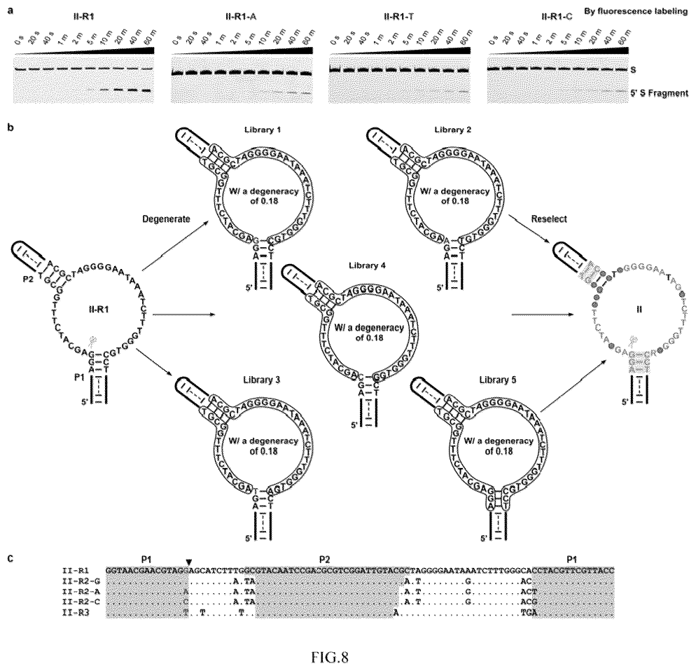

Post-reselection analysis revealed one mutant, named II-R2, cleaved with a 1-h yield over 85% for 5′-G/A/C\hat{ } but inadequate (63%) for 5-T\hat{ }, and a second mutant, named II-R3, whose 1-h yield reached ˜88% for 5′-T\hat{ }(FIG. 1c-d&f, FIGS. 8e& 9a). Comprehensive covariation experiments confirmed that the nucleotides in the stem upstream of 5′-G/A/C\hat{ } for II-R2 and 5′-T\hat{ } for II-R3 are programmable (FIG. 9b-e). Thus the combinatorial usage of II-R2 and II-R3 as DNAzyme 2 can robustly generate 5′ cleavage products with customizable 3′ termini (5′-NN\hat{ }).

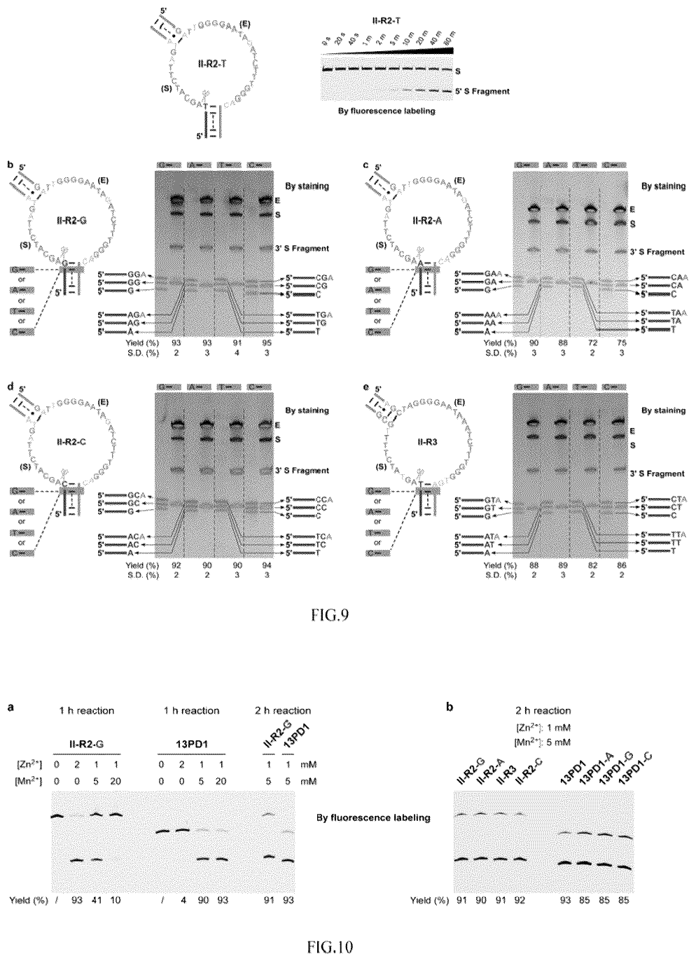

Consequently, pairing II-R2/3 to 13PD1 for double cutting should generate DNA fragments with customized 5′-to-3′ sequences (5′-NN . . . NN-3′) (FIG. 1a). Considering that the metal ion dependency of these enzymes is different, we identified a compatible buffer condition (50 mM HEPES, μl 7.0, 1 mM Zn²⁺ and 5 mM Mn²⁺), in which each DNAzyme can self-cleave over 85% in 2 h at 37° C. (FIG. 10), so that simultaneous cutting of the two enzymes can be achieved in a one-pot reaction. Based on these findings, we were able to program the pair of proper DNAzyme 1&2 to flank arbitrary sequences of target oligos (FIG. 11), synthesized the whole as pseudogene fragments, and amplified them via the p3024 phagemid vector (a pBluescript variant) for PECAN-oligo production (see Methods, FIG. 1a). Note that because the pair of DNAzymes precisely cleaves the 3′ phosphoester bond, PECAN oligos are born with 5′ phosphate and 3′ hydroxyl termini.

### Example 2 Length, Quantity, Purity, and Sequence Customizability of PECAN Oligos

Recombinant bacteriophages carrying extremely long DNA insertions are prone to yield fragmented byproducts when replicated in vivo. Our experiments revealed that with insertions of <7,000 base-pair (bp), the recombinant p3024 can be efficiently amplified in Escherichia coli to consistently generate byproduct-free ss-phagemid. By using shaker-flask cultures, we routinely gathered milligrams of ss-phagemid. The quantity can be further elevated to the gram level with a 10 liter laboratory fermenter (FIG. 12a), consistent with the previous reports on phage production. During autoprocessing post amplification, the paired usage of II-R2/3 and 13PD1 displayed robust effects in excising a series of oligos of different lengths. ranging from ˜70mer to 6,750mer, with >85% yields off the ss-phagemid precursors for collection (FIG. 12b).

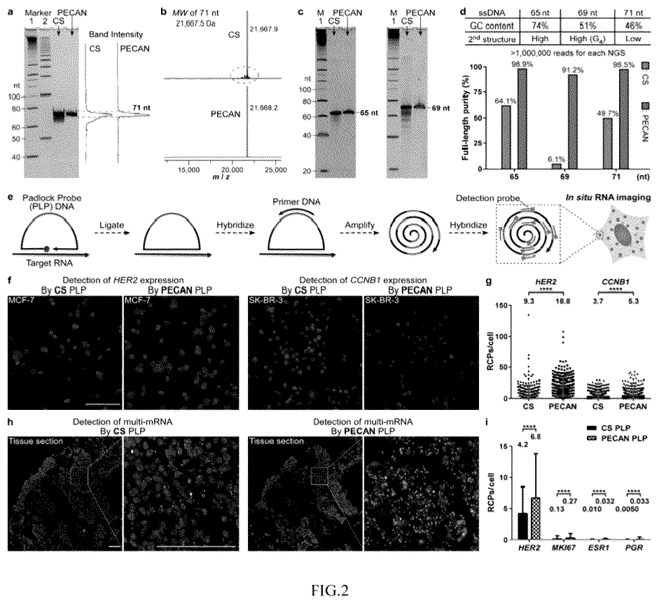

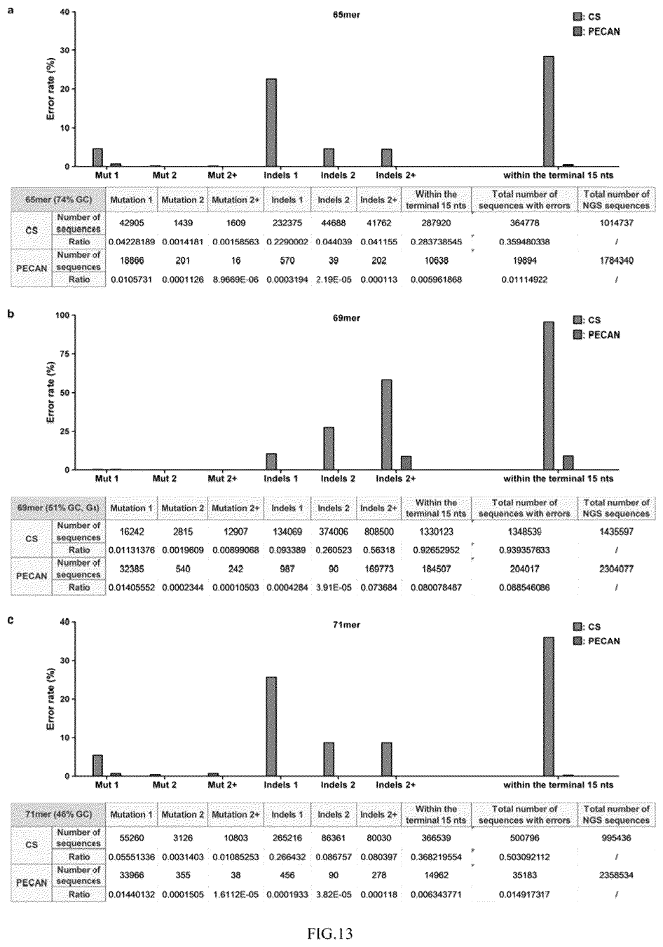

PECAN recruits the bacterial replication machinery and the self-catalytic DNAzyme to produce DNA oligos massively and efficiently. The high fidelity of the former and the high specificity of the latter should also in principle guarantee the purity of the oligo products. To verify that, we compared oligos produced by PECAN to chemical synthesis (CS) (FIG. 2a-d). The chosen representatives include a 65mer, 69mer, and 71mer with 74%, 51%, and 46% GC content, respectively. When subjected to denaturing polyacrylamide gel electrophoresis (dPAGE), all of the three PECAN oligos exhibited a clean and sharp band on the gel, while the CS oligos (PAGE-purified) all yielded a blurry band, suggesting non-ignorable impurity in the latter (FIG. 2a&c). These results were consistent with the data from monoisotopic (exact mass) spectroscopy, wherein the PECAN 71mer displayed a single mass peak of the full-length oligo, while the CS 71mer had many peaks surrounding the major full-length one (FIG. 2b). Using next-generation sequencing (NGS), we thoroughly analyzed the composition of these samples. For CS, the fraction of error-free full-length oligos decreased from 64.1% to 49.7% as the length increased from 65mer to 71mer, and the 69mer telomere sequence had significantly reduced full-length purity of only 6.1%, likely due to its existence of strong secondary structures of G-quadruplex (G₄)(FIG. 2d). Of the errors in CS oligos, single-base indels (insertions or deletions) and mutations accounted for the majority and occurred with higher frequencies at the oligo ends (FIG. 13). Contrarily, error-free full-length oligos occupied >98% of the molecules for both PECAN 65mer and 71mer, regardless of their differences in length and GC content. Even for the extremely error-prone 69mer G4 sequence, PECAN ensured >90% purity (FIG. 2d). Thus PECAN can achieve single-base accuracy for oligo production.

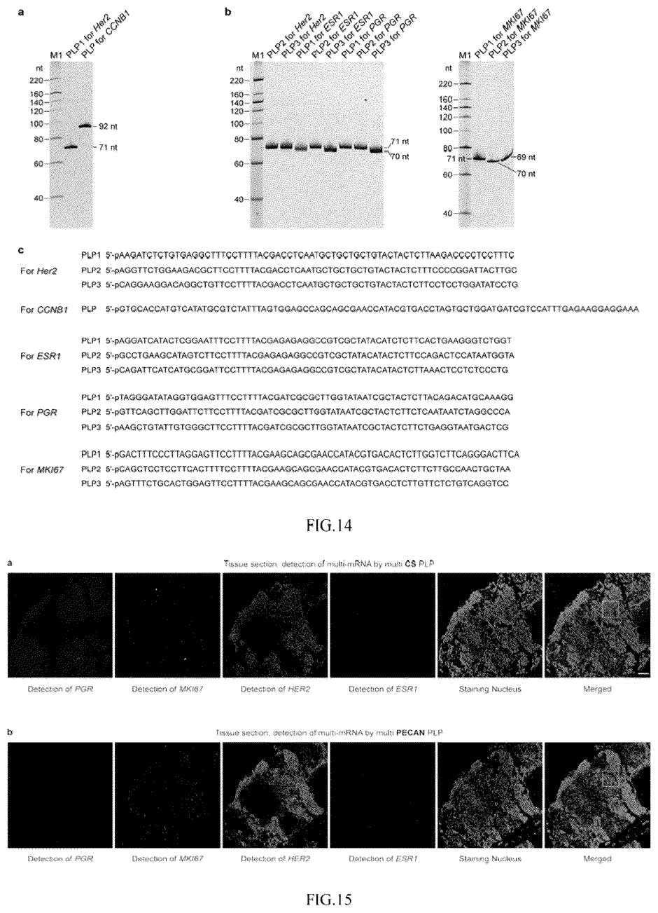

Besides the three representatives, through PECAN we successfully produced diverse DNA oligos with distinct sequence identities, especially at the termini (FIG. 14), and with various sequence lengths, from dozens to hundreds to thousands of nucleotides (FIG. 3e, 4a), demonstrating the decent customizability of PECAN.

### Example 3 Application to RNA In Situ Detection

The 60-100mer DNA oligos can be engineered as the widely used padlock probes (PLPs) in single molecule fluorescence in situ hybridization, a powerful technique to study gene expression in single cells. PLP in combination with rolling-circle amplification (RCA) can generate clonally amplified rolling-circle products (RCPs) at high density in preserved tissue and cells for detection (FIG. 2e), genotyping, and sequencing of individual RNA molecules In situ. Currently, CS oligos are the main source of PLP, and little is known to whether the detection efficiency, positively correlated with the RNA resolution, is affected by oligos' quality. With much purer PECAN oligos (FIG. 2a-d), we launched a preliminary exploration into that.

In the breast carcinoma cell line MCF-7, expression of HER2 mRNA is reported at a low level (nTPM=4.8) according to the human protein atlas database (www.proteinatlas.org). With CS PLP, we recorded a mean number of RCPs per MCF-7 cell to be 9.3 (n=283). As switching to PECAN PLP instead (FIG. 14), the detected number increased to 18.8 (n=316), which corresponds to an improvement in detection efficiency by 102% (FIG. 2f-g). Similarly, in a second cell line SK-BR-3 with low expression of CCNB1. the detected mean number of RCPs per cell increased from 3.7 (n=787) by CS PLP to 5.3 (n=639) by PECAN PLP, wherein the efficiency was enhanced by 43%(FIG. 2f-g).

To further investigate the efficiency during multiplexed in situ RNA detection on routine clinical sample, we moved to formalin-fixed, paraffin-embedded (FFPE) breast cancer tissue sections, which had been classified as HER2-positive by immunohistochemistry (IHC) in the diagnostics laboratory, and simultaneously examined four transcripts on two consecutive slides, with CS PLP for one and PECAN PLP for the other (FIG. 2h-i). These RNAs include HER2, MK167, ESR1, and PGR, corresponding to the conventionally IHC tested breast cancer biomarkers Her-2, Ki-67, ER, and PR, respectively. Both PLPs detected relatively high expression of HER2 RNA in the tissue sections, with PECAN PLP displaying 62% efficiency more than CS PLP (FIG. 2h-i). Meanwhile, as expected, both PLPs detected very low expression of MK167, ESR1, and PGR in the HER2-positive tissue sections (FIG. 15). However, on these three RNAs, PECAN PLP possessed >100% enhancement in detection efficiency over CS PLP, clearly shown by the statistics analysis (P<0.0001) (FIG. 2i). In summary, our initial studies confirmed that as probes, PECAN oligos can steadily improve the RNA detection efficiency in tissue and cells. The result is consistent with the purity of >98% vs <65% between the routine PECAN and CS oligos (FIG. 2d).

### Example 4 Application to Large-Fragment Capture for Targeted Sequencing

Specific capture of long, multi-exon-sized (˜200-1,000 bp) genomic regions can accurately preserve a sample's genome information for functional analysis of gene products and regulatory elements by targeted sequencing. For this purpose, long padlock or long-adapter single-strand oligonucleotide (LASSO) probe, usually 300-500mer in length, has been developed as the next-generation molecular inversion probe to overcome the persistence length (stiffness) of long dsDNA during the capturing process (FIG. 3b). However, current synthetic methods cannot efficiently produce such long oligomers, thus hampering our understanding of the functional significance of genes from the high-quality sequences generated by large-fragment capture.

We believe that PECAN provides an ultimate solution to LASSO probes. As an initial proof of concept, we produced LASSO probes by PECAN to analyze alternative splicing in different cell lines. The gene S100P transcribes in a single isoform (NCBI accession: NM_005980.3) in hepatoma 97L but not normal liver (NL) cells, and CYP24A1 has two validated splicing variants, tv1 (NCBI accession: NM_000782.5) and tv2 (NCBI accession: NM_001128915.2), with the former expressed only in 97L and the latter in both (FIG. 3a). To accurately display these transcription differences, we designed LASSO probe 1 (335mer) and probe 2 (550mer) for specific targeting to S100P and CYP24A1 tv1, respectively. After gap filling and ligation, a 401-bp fragment in S100P and a 749-bp fragment in CYP24A1 tv1 were supposed to be captured for targeted amplification and sequencing (FIG. 3b).

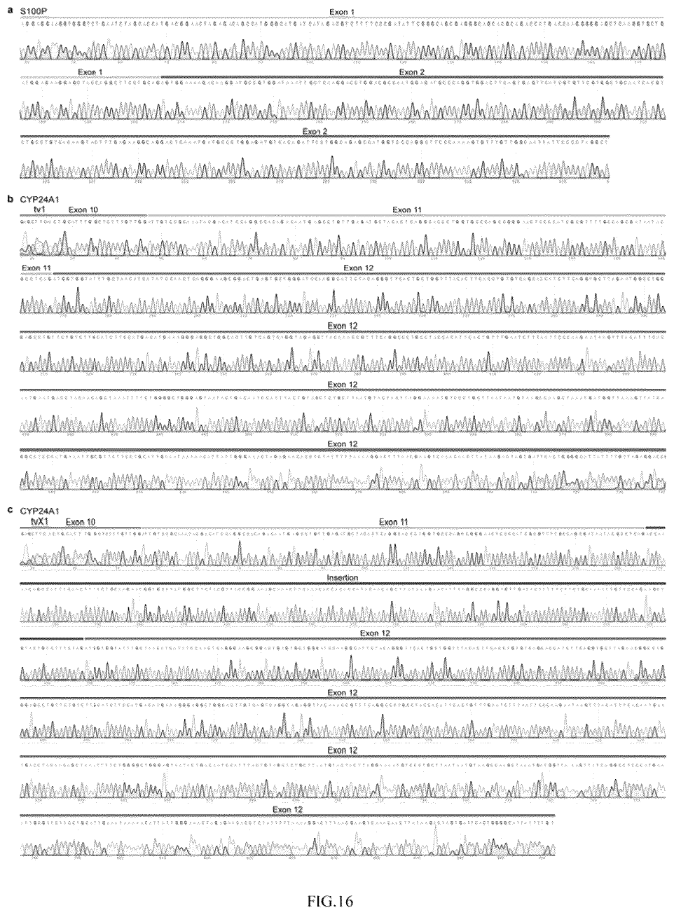

By dPAGE, we firstly confirmed the purity and length of PECAN LASSO 1&2 (FIG. 3c). After amplification, the capture of a fragment with the expected length from 97L but not NIL by PECAN LASSO 1 was revealed on the agarose gel (FIG. 3d). This fragment was later demonstrated to be from S100P by sequencing (FIG. 3e, FIG. 16a). Interestingly, for the capture by PECAN LASSO 2, other than the expected fragment, a second one (referred by *) with a size of ˜200 bp longer also showed up from 97L but not NL (FIG. 3d). Sequencing data verified that the expected fragment belonged to CYP24A1 tv1, and the unexpected one actually came from CYP24A1 tvX1 (NCBI accession: XM_005260304.5) (FIG. 3e, FIG. 16b-e), another isoform of CYP24A1 that had been predicted by bioinformatics but not been experimentally validated in 971 yet. CYP24A1 tvX1 differs from CYP24A1 tv1 only in a 172-bp insertion between exon 11&12, thus capable of being captured by PECAN LASSO 2 that was originally designed to hybridize with exon 10& 12 for specific capture of CYP24A1 tv1 (FIG. 3b). By using PECAN LASSO 2, we further screened a series of hepatoma cell lines to explore splice isoforms of CYP24A1, and found that CYP24A1 tv1 & tvX1 were both expressed in 97L, 97H, LM3, and HepG2 but not in Hu7 cells, demonstrating the feasibility of PECAN oligos.

### Example 5 Application to Homology-Directed Genome Editing

The rapid development of genome editing technologies, particularly the CRISPR/Cas9 system, has enabled efficient generation of knockout (KO) cell and mouse models through error-prone nonhomologous end joining. However, the efficiency of precise sequence insertion, e.g., knock-in (KI) of reporters or recombinases. and precise sequence replacement, e.g., conditional KO of alleles with exons flanked by LoxP sites, by using exogenous DNA donors as templates for homology-directed repair (IDR) is very poor, creating an obstacle to generate the most useful genetically engineered models in biomedical research. Recent studies revealed that long, kilo-based ssDNA can be used as an attractive alternative to its equivalent dsDNA HDR template (HDRT) to systematically improve the efficiency of genome editing, but a robust synthetic method to produce such sized oligos is lacking. We speculate that PECAN, with the ability to synthesize arbitrary oligos up to 7,000mer, can resolve the challenging source issue of the HDRT ssDNA.

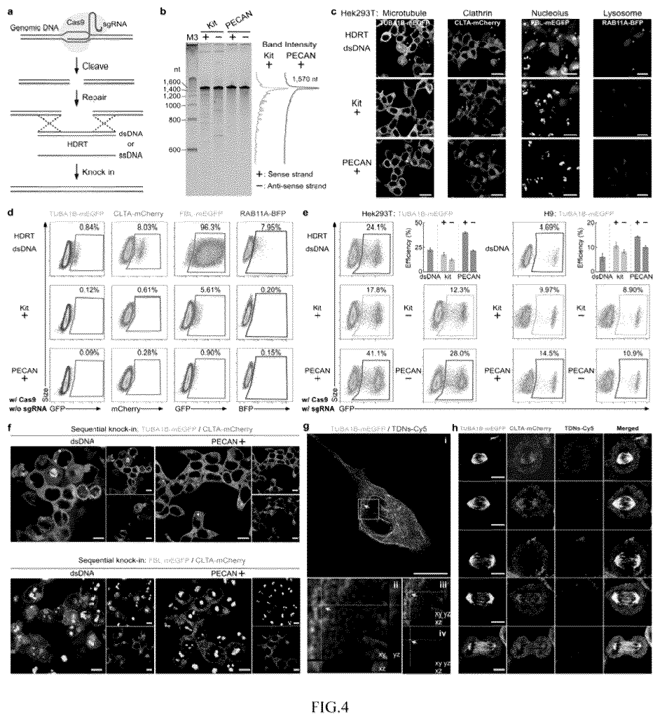

To verify our speculation, we conducted comparative analysis of long DNA donors with equivalent sequences but in different forms, including dsDNA prepared by PCR amplification and ssDNA prepared by kit or PECAN, to introduce fluorescent reporters into human cell lines (FIG. 4). We chose the Guide-it™ Long ssDNA Strandase Kit to selectively degrade one of the two strands in dsDNA for Kit ssDNA production. Based on a previous report, we designed HDRTs with ˜400mer homology arms to maximize the KI efficiency, resulting in the preparation of a series of ˜1,500 bp or ˜1,500 nt HDRTs in order to fuse a fluorescent tag (˜700 nt) to the house-keeping gene TUBA1B, CLTA, FBL, or RAB11A in Hek293T cells.

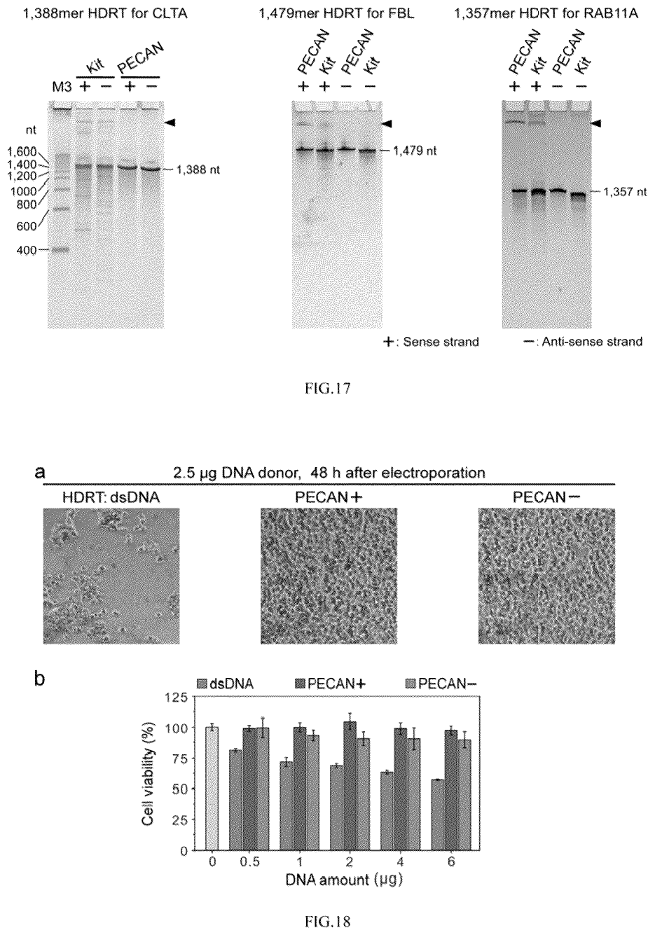

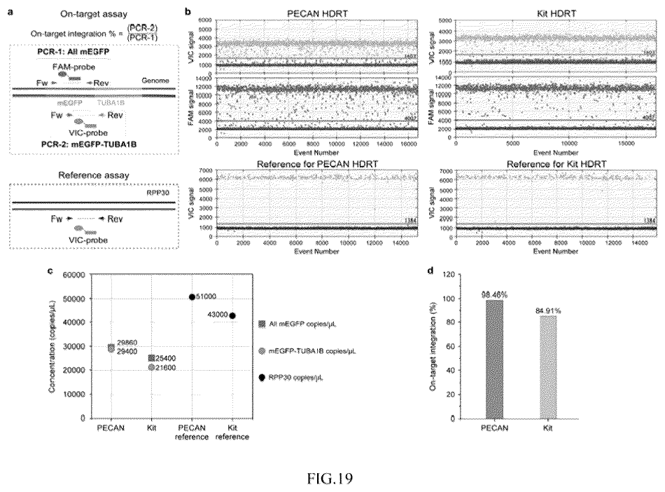

Analysis by dPAGE confirmed much purer oligo products of all ˜1,500mer MDRTs by PECAN over Kit (FIG. 4b, FIG. 17). When electroporated into Hek293T cells, PECAN HDRTs caused negligible loss in viability (FIG. 18). After fluorescence activated cell sorting, live-cell imaging displayed apparent off-targeting for all KIs by dsDNA HDRTs and the KI at FBL locus by Kit HDRT, in contrast to invisible off-targeting for all KIs by PECAN HDRTs, suggesting that PECAN HDRTs is the most specific KI donor among the three (FIG. 4c). These results were consistent with a quantitative off-target measurement by omitting the single guide RNA (sgRNA) from the CRISPR system, wherein PECAN HDRTs led to <1% off-target integration for all including the FBI. locus, while at this locus >5% and >95% oft-targeting were detected in the KIs with Kit and dsDNA HDRTs, respectively (FIG. 4d). We inferred that the low off-target rate with PECAN HDRTs benefitted from the high quality of these DNAs, in particular the absence of dsDNA fragments in the oligo products as compared to Kit and dsDNA HDRTs, because dsDNA donors can lead to high rate of non-specific KI through potential non-homologous integration of dsDNA at random double-strand breaks.

Next, we chose TUBA1B gene that displayed <1% off-targeting with all three types of HDRTs (FIG. 4d) to evaluate the apparent KI efficiency (percentage of the GFP-positive cells). In Hek293T cells, we detected that PECAN HDRTS led to ˜28-41% GFP KI, >2-fold more efficient than Kit HDRTs and surpassing that by dsDNA HDRTs too, regardless of with the sense or antisense strand (FIG. 4e). In KI-less-prone H9 cells, we achieved 14.5% apparent efficiency with a PECAN HDRT, and the efficiency decreased from PECAN to Kit to dsDNA HDRT, with a 3-fold fluctuation between the best and the least (FIG. 4e). Through a digital droplet PCR assay, we further characterized that among GFP-positive Hek293T cells, the on-target GFP frequency (specificity) at the TUBA1B locus reached 98.46% with PECAN vs 84.91% with Kit HDRT (FIG. 19).

PECAN donors' high KI efficiency and specificity allowed us to readily construct high-quality cell models carrying double KI of fluorescent reporters (FIG. 4f). With these stain-free cell models, we conducted a preliminary study on mammalian cell internalization of tetrahedral DNA nanostructures (TDNs), which had been widely used in DNA nanotechnology as carriers for drug delivery. Under confocal microscopy, we observed the colocalization of cyanine-5 (Cy5) labeled TDNs with GFP-tagged microtubules (FIG. 4g), supporting a microtubule-dependent transportation of TDNs in cells. Interestingly, we also found the overlapping of Cy5-TDNs with RFP-tagged clathrins during different phases of the cell cycle (FIG. 4h), implying that other than the reported uptake by caveolin, clathrin also engaged in the endocytosis of TDNs, likely through the clathrin-microtubule pathway. As further work is needed to completely unveil TDNs' internalization mechanism, it is no doubt that the precise KI of reporters with PECAN HDRTs, revealed by the distinct sub-cellular localization of each of the reporter-fusion proteins (FIG. 4h), can provide clean and clear endogenous references for high-quality cell imaging.

### Example 6 Application to DNA-Based Data Storage

The superior density, durability, longevity, and energy efficiency have made DNA also an intriguing medium to store digital information in our Big Data era. Several architectures and platforms for DNA-based storage have been established, using pools of <300mer oligos achievable through chemical or enzymatic synthesis. But the accumulated cost in error correction, sub-segment re-assembly, etc., over larger data payloads makes current DNA-based storage systems uncompetitive with existing flash technologies. Nevertheless, such status quo may be changed with the emergence of PECAN, through which extremely long (˜7,000 mer), high-quality oligos can be provided as huge building blocks to lower protocol overhead in DNA-based storage systems.

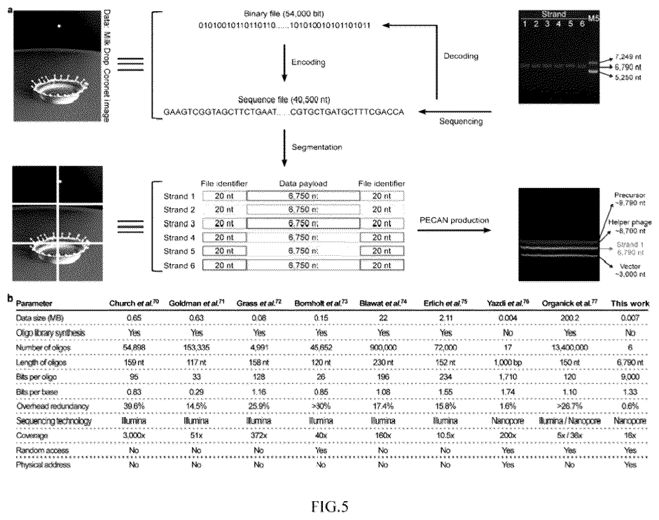

As a proof of concept, we selected the 6.75 KB ‘Milk Drop Coronet’ JPG image for storage by super long PECAN oligos (FIG. 5a). Based on a reported coding strategy, we converted this image file into an 81,000-bit bitstream, which can be encoded by a 40,500-nt DNA sequence. Such coding provided a net information density of 1.33 bit/nt, comparable to that of most existing strategies (FIG. 5b). We then segmented the 40,500-nt sequence into six 6,790-nt oligos, each containing a 6,750-nt codeword domain and a unique, strand-specific 40-nt address for indexing. After PECAN production and oligo quality check, each 6,790mer strand was individually stockpiled with a physical address (FIG. 5a). To retrieve the image file, we sampled each individual oligo and created an oligo mixture for sequencing by the Oxford PromethION Nanopore sequencer. Within minutes, the sequencing data was generated and decoded, resulting in the recovery of the image with 100% accuracy.

Several advantages can be seen for the PECAN oligo-assisted storage system (FIG. 5b). First, the long, predominant payload region on PECAN oligos ensures the system's coding efficiency by reducing overhead redundancy (percentage of the non-payload codes) to as low as 0.6%, nearly 20-60 folds lower than that of most known DNA-based storage systems. Second, PECAN oligos can be produced in massive amounts (gram level by a laboratory fermenter, FIG. 12) with high fidelity (inherited from the faithful in view replication), therefore supporting a practical and economical writing & reading mode, that is write-once, yield-numerous-data-copies, and read-numerous in a PCR-independent manner at lower cost per copy of data and lower error rate. Third, comparing to oligo library synthesis, although synthesis of oligos individually by PECAN initially increases the production cost, the thus acquired physical address for each oligo provides convenience for random access, making possible the PCR-independent selective reading of data, a benefit that certainly amortizes the cost. Last but not least, the ˜7,000mer super long PECAN oligos allow us to fully exploit the state-of-the-art nanopolish platforms to reduce sequencing cost.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and G on 3′ side of said cleavage site.

2. The system of claim 1, wherein said system comprises one or more catalytic nucleic acids, and said catalytic nucleic acids comprise one or more said catalytic domains.

3. The system of claim 2, wherein said system comprises one or more substrate nucleic acids, and said substrate nucleic acids comprise one or more said substrate domains.

4. The system of claim 2, wherein one or more said catalytic nucleic acids and one or more said substrate nucleic acids are separate and/or conjugated.

5. The system of claim 1, wherein said system further comprises one or more binding domains, and said binding domains flank and/or is within said catalytic domains and/or said substrate domains.

6. The system of claim 5, wherein said catalytic nucleic acids comprise one or more binding domain A, said substrate nucleic acids comprise one or more binding domain B, and said binding domain A is capable of binding to said binding domain B.

7. The system of claim 5, wherein said catalytic nucleic acids comprise binding domain A-5 on 5′ side of said catalytic domains and binding domain A-3 on 3′ side of said catalytic domains, said substrate nucleic acids comprise binding domain B-5 on 5′ side of said substrate domains and binding domain B-3 on 3′ side of said substrate domains, and said binding domain A-5 is complementary to said binding domain B-3 and/or said binding domain A-3 is complementary to said binding domain B-5.

8. The system of claim 1, wherein said 13PD comprise 13PD1, 13PD2, 13PD3, 13PD4, and/or the mutant thereof.

9. The system of claim 1, wherein said catalytic domains comprise sequence of SEQ ID NO: 17.

10. The system of claim 1, wherein said catalytic domains comprise nucleic acid hydrolysis activity.

11. The system of claim 1, wherein said substrate domains comprise sequence of SEQ ID NO: 18 (actgcn, wherein n is a, c or g).

12. The system of claim 1, said substrate domains comprise A, C, or G on 3′ end of said substrate domains.

13. A nucleic acid, comprising one or more catalytic domains and one or more substrate domains, wherein said catalytic domains comprise 13PD catalytic domain sequence, said catalytic domains cleave said substrate domains at a cleavage site, and said substrate domains comprise a base selected from the group consisting of A, C, and G on 3′ side of said cleavage site.

14. The nucleic acid of claim 13, wherein said nucleic acid further comprises one or more binding domains, and said binding domains flank and/or is within said catalytic domains and/or said substrate domains.

15. The nucleic acid of claim 14, wherein said nucleic acids comprise one or more binding domain A flanking said catalytic domains, said nucleic acids comprise one or more binding domain B flanking said substrate domains, and said binding domain A is capable of binding to said binding domain B.

16. The nucleic acid of claim 14, wherein said nucleic acids comprise binding domain A-5 on 5′ side of said catalytic domains and binding domain A-3 on 3′ side of said catalytic domains, said nucleic acids comprise binding domain B-5 on 5′ side of said substrate domains and binding domain B-3 on 3′ side of said substrate domains, and said binding domain A-5 is complementary to said binding domain B-3 and/or said binding domain A-3 is complementary to said binding domain B-5.

17. The nucleic acid of claim 13, wherein said 13PD comprise 13PD1, 13PD2, 13PD3, 13PD4, and/or the mutant thereof.

18. The nucleic acid of claim 13, wherein said catalytic domains comprise sequence of SEQ ID NO: 17.

19. The nucleic acid of claim 13, wherein said catalytic domains comprise nucleic acid hydrolysis activity.

20. The nucleic acid of claim 13, wherein said substrate domains comprise sequence of SEQ ID NO: 18 (actgcn, wherein n is a, c or g).

21. The nucleic acid of claim 13, said substrate domains comprise A, C, or G on 3′ end of said substrate domains.

22. A vector, comprising the nucleic acid of claim 13.

23. A cell, comprising the nucleic acid of claim 13.

24. A composition, comprising the nucleic acid of claim 13.

25. A kit, comprising, the nucleic acid of claim 13.

26. A method of preparing a product, comprising providing the system of claim 1.

27. A product prepared according to the method of claim 26.

28. The product of claim 27, said product comprises nucleic acid.

29. A combination, comprising providing a condition comprising about 1 to 2 mM Zn²⁺, and about 5 to 20 mM Mn²⁺.

30. The combination of claim 29, comprising providing a condition comprising about 1 mM Zn²⁺, and about 5 mM Mn²⁺.

31. The combination of claim 29, comprising about 1 mM Zn²⁺, and about 5 mM Mn²⁺.

32. A method of preparing a product, comprising providing the combination of claim 29 and providing 5′ nucleic acid cutter and 3′ nucleic acid cutter.

33. The method of claim 32, said 5′ nucleic acid cutter comprises DNAzyme I capable of generating 3′ cleavage product.

34. The method of claim 32, said 5′ nucleic acid cutter is on 5′ side of said product.

35. The method of claim 32, said 5′ nucleic acid cutter comprises 13PD and mutant thereof.

36. The method of claim 32, said 3′ nucleic acid cutter comprises DNAzyme II capable of generating 5′ cleavage product.

37. The method of claim 32, said 3′ nucleic acid cutter is on 3′ side of said product.

38. The method of claim 32, said 3′ nucleic acid cutter comprises II-R1 and mutant thereof.

39. The method of claim 32, said 3′ nucleic acid cutter comprises II-R1a, II-R1b, II-R1c, II-R1d, and mutant thereof.

40. A product prepared according to the method of claim 32.

41. The product of claim 40, said product comprises nucleic acid.

42. A method of nucleic acid detection, comprising providing the product of claim 40.

43. A method of sequencing, comprising providing the product of claim 40.

44. A method of genetic engineering, comprising providing the product of claim 40.

45. A method of data storage, comprising providing the product of claim 40.


SEQUENCE LISTING (30 sequences)

SEQ ID NO: 1 - DNA, 91 bp, synthetic construct
  misc_feature 1..91: note = IDT1 or 5
  source 1..91: mol_type = other DNA
gagtgctacgaacgtaggagcatctttggcgtacaagcgaagcttgtacgctaggggaataaatctttgggtgcctacgttcgtaggactc

SEQ ID NO: 2 - DNA, 91 bp, synthetic construct
  misc_feature 1..91: note = IDT2
  source 1..91: mol_type = other DNA
gagtgctacgaacgtagaagcatctttggcgtacaagcgaagcttgtacgctaggggaataaatctttgggtgtctacgttcgtaggactc

SEQ ID NO: 3 - DNA, 91 bp, synthetic construct
  misc_feature 1..91: note = IDT3
  source 1..91: mol_type = other DNA
gagtgctacgaacgtagtagcatctttggcgtacaagcgaagcttgtacgctaggggaataaatctttgggtgactacgttcgtaggactc

SEQ ID NO: 4 - DNA, 91 bp, synthetic construct
  misc_feature 1..91: note = IDT4
  source 1..91: mol_type = other DNA
gagtgctacgaacgtagcagcatctttggcgtacaagcgaagcttgtacgctaggggaataaatctttgggtggctacgttcgtaggactc

SEQ ID NO: 5 - DNA, 15 bp, synthetic construct
  misc_feature 1..15: note = Primer 1
  source 1..15: mol_type = other DNA
gagtgctacgaacgt

SEQ ID NO: 6 - DNA, 15 bp, synthetic construct
  misc_feature 1..15: note = Primer 2-5
  source 1..15: mol_type = other DNA
aaaaaaaaaaaaaaa

SEQ ID NO: 7 - DNA, 15 bp, synthetic construct
  misc_feature 1..15: note = Primer 2-3
  source 1..15: mol_type = other DNA
gagtcctacgaacgt

SEQ ID NO: 8 - DNA, 71 bp, synthetic construct
  misc_feature 1..71: note = 13PD1 or -A-C-G enzyme sequence
  source 1..71: mol_type = other DNA
gtcgccatctcttctataccgggcaactattgcctcgtcatcgctattttctgcgatagtgagtcgtatta

SEQ ID NO: 9 - DNA, 36 bp, synthetic construct
  misc_feature 1..36: note = 13PD1 substrate sequence
  source 1..36: mol_type = other DNA
taatacgactcactatactgctgaagagatggcgac

SEQ ID NO: 10 - DNA, 36 bp, synthetic construct
  misc_feature 1..36: note = 13PD1-A substrate sequence
  source 1..36: mol_type = other DNA
taatacgactcactatactgcagaagagatggcgac

SEQ ID NO: 11 - DNA, 36 bp, synthetic construct
  misc_feature 1..36: note = 13PD1-C substrate sequence
  source 1..36: mol_type = other DNA
taatacgactcactatactgccgaagagatggcgac

SEQ ID NO: 12 - DNA, 36 bp, synthetic construct
  misc_feature 1..36: note = 13PD1-G substrate sequence
  source 1..36: mol_type = other DNA
taatacgactcactatactgcggaagagatggcgac

SEQ ID NO: 13 - DNA, 110 bp, synthetic construct
  misc_feature 1..110: note = 13PD1
  source 1..110: mol_type = other DNA
gtcgccatctcttctataccgggcaactattgcctcgtcatcgctattttctgcgatagtgagtcgtattattttaatacgactcactatactgctgaagagatggcgac

SEQ ID NO: 14 - DNA, 110 bp, synthetic construct
  misc_feature 1..110: note = 13PD1-A
  source 1..110: mol_type = other DNA
gtcgccatctcttctataccgggcaactattgcctcgtcatcgctattttctgcgatagtgagtcgtattattttaatacgactcactatactgcagaagagatggcgac

SEQ ID NO: 15 - DNA, 110 bp, synthetic construct
  misc_feature 1..110: note = 13PD1-C
  source 1..110: mol_type = other DNA
gtcgccatctcttctataccgggcaactattgcctcgtcatcgctattttctgcgatagtgagtcgtattattttaatacgactcactatactgccgaagagatggcgac

SEQ ID NO: 16 - DNA, 110 bp, synthetic construct
  misc_feature 1..110: note = 13PD1-G
  source 1..110: mol_type = other DNA
gtcgccatctcttctataccgggcaactattgcctcgtcatcgctattttctgcgatagtgagtcgtattattttaatacgactcactatactgcggaagagatggcgac

SEQ ID NO: 17 - DNA, 41 bp, synthetic construct
  misc_feature 1..41: note = 13PD1 catalytic domain
  source 1..41: mol_type = other DNA
tataccgggcaactattgcctcgtcatcgctattttctgcg

SEQ ID NO: 18 - skipped

SEQ ID NO: 19 - DNA, 16 bp, synthetic construct
  misc_feature 1..16: note = TUBA1B Forward
  source 1..16: mol_type = other DNA
aggaaggccttcccgg

SEQ ID NO: 20 - DNA, 26 bp, synthetic construct
  misc_feature 1..26: note = TUBA1B Reverse
  source 1..26: mol_type = other DNA
catagttattggcagcatcttccttg

SEQ ID NO: 21 - DNA, 19 bp, synthetic construct
  misc_feature 1..19: note = CLTA Forward
  source 1..19: mol_type = other DNA
ggcagcacttccgcctccc

SEQ ID NO: 22 - DNA, 24 bp, synthetic construct
  misc_feature 1..24: note = CLTA Reverse
  source 1..24: mol_type = other DNA
aaggagcacgagtcacacagaagc

SEQ ID NO: 23 - DNA, 23 bp, synthetic construct
  misc_feature 1..23: note = FBL Forward
  source 1..23: mol_type = other DNA
gagaaaaacagcccaaagccctg

SEQ ID NO: 24 - DNA, 22 bp, synthetic construct
  misc_feature 1..22: note = FBL Reverse
  source 1..22: mol_type = other DNA
agcaaaatggcgaccacaacaa

SEQ ID NO: 25 - DNA, 23 bp, synthetic construct
  misc_feature 1..23: note = RAB11A Forward
  source 1..23: mol_type = other DNA
ctaggagttccaggactcagttt

SEQ ID NO: 26 - DNA, 24 bp, synthetic construct
  misc_feature 1..24: note = RAB11A Reverse
  source 1..24: mol_type = other DNA
gacgatgtgggagaaggcagtccg

SEQ ID NO: 27 - DNA, 1570 bp, synthetic construct
  misc_feature 1..1570: note = mEGFP-TUBA1B HDRT
  source 1..1570: mol_type = other DNA
aggaggccttcccggtttaggatgggaaggtaacattcattaaaagcaacgtagactatagtgtagctgttctcaaaagtagtacatcttagaaaaggatctttagaaaagatcgctttagaaaaggaaattcgttttcagattacgtgagtagcctaggtaacacagccagacctcatctccacaaaaaaaatgaaaaaattagccagcttggtggtctgtgcctgtggtcccagctgctccagaggctgaggtggggggatgactggagcctaggctgcagtgagcctagatggcatcactgcactcaagcctgggcgacagaccttatctctaaaaaaataaagattgcatgagtattttgttccacttgacagtcatcaatagattggtttaaattgtgatatcttttttacttaccgcaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagggaggttcaggaggcagcgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattggtgagttgacctcagtaacctgagatcccaggatgctgggacaggaggtctgtccaggggcttctcttgtcactcactcactccctccgtccttctctccctcctccagatgaagttcgcactggcacctaccgccagctcttccaccctgagcagctcatcacaggcaaggaagatgctgccaataactatg

SEQ ID NO: 28 - DNA, 1480 bp, synthetic construct
  misc_feature 1..1480: note = FBL-mEGFP HDRT
  source 1..1480: mol_type = other DNA
gagaaaaacagcccaaagccctgttgtagacattagtcctttctcctctttaggccaactgcattgactccacagcctcagccgaggccgtgtttgcctccgaagtgaaaaagatgcaacaggagaacatgaagccgcaggagcagttgacccttgagccatatgaaagagaccatgccgtggtcgtgggagtgtacaggtgagcaggggcccagcaatacaccaagacagacatctctgtcccttgcaccccgagtgccatgatcctggggaccctccttcatcacctatcttcctctcacaggccacctcctaaagtgaagaacaagcccaacagcgccgtggacggcaccgccggccccggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaatgaagttcagccctgagcggattgcgagagatgtgtgttgatactgttgcacgtgtgtttttctattaaaagactcatccgtctcccatgtctgctgctcattcctccccttgacctgctgacacagggagcacgcacccttggtcaattttgcggggttgggtaaattctcactcggtcacagagcgcatgctccgtttctagctgcctttgcgcagcggcagcctggatttcggttcttgggtgggattggtagctcgctgcgcatgcgtgcaggtaagcggccatctcgcgcaggcggagtgtcagtgtgggtcacgtgaggggagcggagagggagggatgggggcggagtccagggcgtgggggggccggtttgttgtggtcgccattttgct

SEQ ID NO: 29 - DNA, 1390 bp, synthetic construct
  misc_feature 1..1390: note = CLTA-mCherry HDRT
  source 1..1390: mol_type = other DNA
ggcagcacttccgcctcccggggcccgcgcagctcacctccctcacctcccgccctaccccagtcacgagttgttttagggggaccgcccctccacttgctgattgggtagctcctgaaccattgttgtcctctgattggttgttcccttttcggctctgcaacaccgcctagaccgaccggatacacgggtagggcttccgctttacccgtctccctcctggcgcttgtcctcctctcccagtcggcaccacagcggtggctgccgggcgtggtgtcggtgggtcggttggtttttgtctcaccgttggtgtccgtgccgttcagttgcccgccatggctggatcgggtgggactagtggcgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacatgggaaccggtgctggaagtggtgagctggatccgttcggcgcccctgccggcgcccctggcggtcccgcgctggggaacggagtggccggcgccggcgaagaagacccggctgcggccttcttggcgcagcaagagagcgagattgcgggcatcgagaacgacgaggccttcgccatcctggacggcggcgcccccgggccccagccgcacggcgagccgccggggggtccgggtgagagtgcgggcgcgtttggggcgagaggacttgtctggaaactcggtccacagtgggtccgagagcttctgtgtgactcgtgctcctt

SEQ ID NO: 30 - DNA, 1355 bp, synthetic construct
  misc_feature 1..1355: note = RAB11A-BFP HDRT
  source 1..1355: mol_type = other DNA
ctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcggatcgggtgggactagtggcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgtc